(19) United States
(12) Patent Application Publication
(10) Pub. No.: US 2017/0290814 A1
(43) Pub. Date: Oct. 12, 2017

(54) METHODS OF TREATMENT OF CHOLESTASIS AND FIBROSIS

(71) Applicant: GENFIT, Loos (FR)

(72) Inventors: MATHIEU DUBERNET, SANTES (FR); ROBERT WALCZAK, LILLE (FR); PHILIPPE DELATAILLE, RONCHIN (FR)

(21) Appl. No.: 15/457,538

(22) Filed: Mar. 13, 2017

(30) Foreign Application Priority Data
Apr. 11, 2016 (EP) 16305426.5
Apr. 11, 2016 (EP) 16305428.1

(51) Int. Cl. A61K 31/426; A61K 31/428; A61K 31/427; A61K 45/06

(52) U.S. Cl. CPC A61K 31/426; A61K 45/06; A61K 31/428; A61K 31/427

(57) ABSTRACT

The present invention relates compounds useful and their use for treating cholestatic and fibrotic diseases.

### TECHNICAL FIELD

The present invention relates to the field of medicine, in particular to the treatment of cholestatic and fibrotic diseases.

### BACKGROUND

Abnormal and exaggerated deposition of extracellular matrix is the hallmark of all fibrotic diseases, including liver, pulmonary, kidney or cardiac fibrosis. The spectrum of affected organs, the progressive nature of the fibrotic process, the large number of affected persons, and the absence of effective treatment pose an enormous challenge when treating fibrotic diseases.

NTZ, first described in 1975 (Rossignol and Cavier 1975), was shown to be highly effective against anaerobic protozoa, helminths, and a wide spectrum of microbes including both anaerobic and aerobic bacteria (Rossignol and Maisonneuve 1984; Dubreuil, Houcke et al. 1996; Megraudd, Occhialini et al. 1998; Fox and Saravolatz 2005; Pankuch and Appelbaum 2006; Finegold, Molitoris et al. 2009). It was first studied in humans for the treatment of intestinal cestodes (Rossignol and Maisonneuve 1984) and it is now licensed in the United States (Alinia®, Romark laboratories) for the treatment of diarrhea caused by the protozoan parasites Crystosporidium parvum and Giardia intestinalis. NTZ has also been widely commercialized in Latin America and in India where it is indicated for treating a broad spectrum of intestinal parasitic infections (Hemphill, Mueller et al. 2006). The proposed mechanism of action by which NTZ exerts its antiparasitic activity is through the inhibition of pyruvate:ferredoxin oxidoreductase (PFOR) enzyme-dependent electron transfer reactions that are essential for anaerobic metabolism (Hoffman, Sisson et al. 2007). NTZ also exhibits activity against Mycobacterium tuberculosis, which does not possess a homolog of PFOR, thus suggesting an alternative mechanism of action. Indeed, it was shown that NTZ can also act as an uncoupler disrupting membrane potential and intra-organism pH homeostasis (de Carvalho, Darby et al. 2011).

The pharmacological effects of NTZ are not restricted to its antiparasitic activities and in recent years, several studies revealed that NTZ can also confer antiviral activity (Di Santo and Ehrisman 2014; Rossignol 2014). NTZ interferes with the viral replication by diverse ways including a blockade in the maturation of hemagglutinin (influenza) or VP7 (rotavirus) proteins, or the activation of the protein PKR involved in the innate immune response (for a review, see (Rossignol 2014)). NTZ was also shown to have broad anticancer properties by interfering with crucial metabolic and prodeath signaling pathways (Di Santo and Ehrisman 2014)

In an attempt to propose new therapeutic strategies for the treatment of fibrotic diseases, the inventors found that derivatives of compound 2-[(5-nitro-1,3-thiazol-2-yl)carbamoyl]phenyl]ethanoate (Nitazoxanide or NTZ), a synthetic antiprotozoal agent, shows potent antifibrotic properties. Moreover, the evaluation of NTZ in a liver injury model revealed its capacity to reduce circulating bile acid concentration, thus reflecting its potential to treat cholestatic diseases (such as PBC and PSC) in addition to fibrotic diseases. These effects were totally unexpected in view of the properties previously reported for these molecules. Derivatives of NTZ and TZ appear as potential clinical candidates for the treatment of cholestatic diseases and diverse types of fibrotic diseases.

### SUMMARY OF INVENTION

The present invention provides a compound having the following Formula (I):

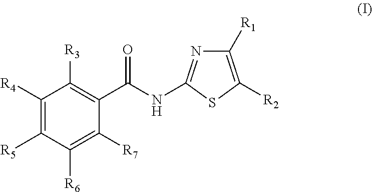

wherein R1 represents a hydrogen atom, a deuterium atom, a halogen atom, a (C6-C14)aryl group, a heterocyclic group, a (C3-C14)cycloalkyl group, a (C1-C6)alkyl group, a sulfonyl group, a sulfoxyde group, a (C1-C6)alkylcarbonyl group, a (C1-C6)alkyloxy, a carboxylic group, a carboxylate group, a nitro group, an amino group, a (C1-C6)alkylamino group, an amido group, a (C1-C6)alkylamido group, a (C1-C6)dialkylamido group,

R2 represents a hydrogen atom, a deuterium atom, a nitro group, a (C6-C14)aryl group, a heterocyclic group, a halogen atom, a (C1-C6)alkyl group, a (C3-C14)cycloalkyl group, a (C2-C6)alkynyl group, a (C1-C6)alkyloxy group, a (C1-C6)alkylthio group, a (C1-C6)alkylcarbonyl group, a (C1-C6)alkylcarbonylamino group, a (C6-C14)arylcarbonylamino group, a carboxylic or carboxylate group, an amido group, a (C1-C6)alkylamido group, a (C1-C6)dialkylamido group, a NH2 group, a (C1-C6)alkylamino group,

or R1 and R2, together with the carbon atoms to which they are attached, form a substituted or unsubstituted 5- to 8-membered cycloalkyl, heterocyclic and aryl group,

R3, R4, R5, R6, and R7, identical or different, represent a hydrogen atom, a deuterium atom, a halogen atom, a hydroxyl group, a (C1-C6)alkylcarbonyl group, a (C1-C6)alkyl group, a (C1-C6)alkyloxy group, a (C1-C6)alkylthio group, a (C1-C6)alkylcarbonyloxy group, a (C6-C14)aryloxy group, a (C6-C14)aryl group, a heterocyclic group, a (C3-C14)cycloalkyl group, a nitro group, a sulfonylaminoalkyle group, a NH2 group, an amino(C1-C6)alkyl group, a (C1-C6)alkylcarbonylamino group, a carboxylic group, a carboxylate group, or a R9 group;

R9 represents a O-R8 group or an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or a moiety of formula (A):

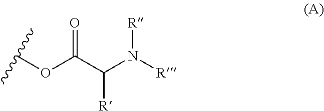

wherein R′ represents a (C1-C6)alkyl, a (C2-C6)alkenyl, a (C2-C6)alkynyl group, a (C3-C14)cycloalkyl group, a (C3-C14)cycloalkyl(C1-C6)alkyl group, a (C3-C14)cycloalkyl(C2-C6)alkenyl group, a (C3-C14)cycloalkenyl group, a (C3-C14)cycloalkenyl(C1-C6)alkyl group, a (C3-C14)cycloalkenyl(C2-C6)alkenyl group, a (C3-C14)cycloalkenyl(C2-C6)alkynyl group;

R″ and R′″, independently, represent hydrogen atom, a (C1-C6)alkyl group, or a nitrogen protecting group;

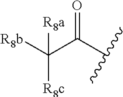

R8 is a hydrogen atom, a deuterium atom, a glucuronidyl group, or a group wherein, R8a, R8b and R8c, identical or different, represent a hydrogen atom or a deuterium atom.

In a particular embodiment, in the compound of formula (I) of the present invention:

an alkyl group may be a substituted or unsubstituted (C1-C6)alkyl group, in particular a substituted or unsubstituted (C1-C4)alkyl group;

an alkynyl group may be a substituted or unsubstituted (C2-C6)alkynyl group;

a cycloalkyl group may be a substituted or unsubstituted (C3-C14)cycloalkyl group;

an alkyloxy group may be a substituted or unsubstituted (C1-C6)alkyloxy group, such as a substituted or unsubstituted (C1-C4)alkyloxy group;

an alkylthio group may be a substituted or unsubstituted (C1-C6)alkylthio group, such as a substituted or unsubstituted (C1-C4)alkylthio group; an alkylamino group may be a (C1-C6)alkylamino group, such as a (C1-C4)alkylamino group;

a dialkylamino group may be a (C1-C6)dialkylamino group, such as a (C1-C4)dialkylamino group;

an aryl group may be a substituted or unsubstituted (C6-C14)aryl group, such as a substituted or unsubstituted (C6-C14)aryl group;

a heterocyclic group may be a substituted or unsubstituted heterocycloalkyl or heteroaryl group.

In a particular embodiment, the compound of the invention is of formula (I) above, with the proviso that when R2 is a nitro group (NO2) and R3 is an acetyl group (CH3CO), then R1, R3, R4, R5, R6 and R7 are not simultaneously a hydrogen atom; with the proviso that when R2 is a nitro group (NO2) and R3 is a hydroxyl group (OH), then R1, R3, R4, R5, R6 and R7 are not simultaneously a hydrogen atom

The present invention also provides pharmaceutical compositions comprising the compounds of formula (I). Accordingly, further objects of the invention include methods of treatment comprising the administration of said compound or pharmaceutical composition for the treatment of cholestatic and fibrotic diseases.

The present invention also provides a compound of formula (I), for use as a medicament.

The present invention also provides a compound of formula (I), for use in a method for the treatment of cholestatic and fibrotic diseases.

### DESCRIPTION OF THE FIGURES AND TABLES

Abbreviations used in the tables, and in the text:
α-SMA alpha Smooth Muscle Actin
ANOVA analysis of variance
BMP Bone Morphogenetic Protein
CCl4 carbon tetrachloride
COL1A1 Collagen, type 1, Alpha 1
DMSO Dimethyl sulfoxide
ECM extracellular matrix
ELISA Enzyme-Linked Immunosorbent Assay
EMT Epithelial-mesenchymal transition
FBS Fetal Bovine Serum
FDA Food and Drug Administration
GDF Growth Differentiation Factors
Hh Hedgehog
hHSC Human Hepatic Stellate Cells
HSC Hepatic Stellate Cells
IC₅₀ Half maximal Inhibitory Concentration
MMP2 Matrix Metallopeptidase 2
MMP9 Matrix Metallopeptidase 9
μl microliter
NHLF Normal Human Lung Fibroblasts
NTZ Nitazoxanide
PBC Primary Biliary Cholangitis
PBS Phosphate Buffer Saline
pMol: picomoles
PSC Primary Sclerosing Cholangitis
rhFGF recombinant human basic Fibroblast Growth Factor
RT Reverse Transcriptase
SD standard derivation
SEM standard error of the mean
SmBM Smooth Muscle cell Basal Medium
SteCGS Stellate Cell Growth Supplement
STeCM Stellate Cell Medium
TBA Total Bile Acids
TGFβ1 Tumor Growth Factor beta 1
TGFBRI TGFb type I receptor
TGFBRII TGFb type II receptor
THBS1 Thrombospondine 1
TMB Tetramethylbenzidine
TZ Tizoxanide

FIG. 1: RM-5061 inhibits TGFβ1-induced expression of α-SMA protein in human HSC.

Serum-deprived HSC were preincubated for 1 hour with RM-5061 before the activation with the profibrogenic cytokine TGFβ1 (1 ng/ml). After 48 hours of incubation, the α-SMA content was measured by ELISA. The obtained values were transformed into percentage inhibition over TGFβ1 control. Data are presented as mean (quadruplicates)±standard error of the mean (SEM). Statistical analyses were performed by one-way ANOVA followed by Bonferroni post-hoc tests, using Sigma Plot 11.0 software. [*: p<0.05; **: p<0.01; ***: p<0.001 (comparison versus TGFβ1 1 ng/mL group)]. The curve fitting and the calculation of half maximal inhibitory concentration (IC₅₀) were performed with XLFit software 5.3.1.3.

FIG. 2: RM-5061 inhibits TGFβ1-induced expression of α-SMA protein in NHLF.

Serum-deprived NHLF were preincubated for 1 hour with RM-5061 before the activation with the profibrogenic cytokine TGFβ1 (1 ng/ml). After 48 hours of incubation, the expression of α-SMA was measured by ELISA. The obtained values were transformed into percentage inhibition over TGFβ1 control. Data are presented as mean (quadruplicates)±standard error of the mean (SEM). Statistical analyses were performed by kruskal-Wallis test followed by Dunn's multiple comparison tests, using GraphPad Prism 5.02 software. [*: p<0.05; **: p<0.01; ***: p<0.001 (comparison versus TGFβ1 1 ng/mL group)]. The curve fitting and the calculation of half maximal inhibitory concentration (IC₅₀) were performed with XLFit software 5.3.1.3.

FIG. 3: Cpd.5 inhibits TGFβ1-induced expression of α-SMA protein in human HSC.

Serum-deprived HSC were preincubated for 1 hour with Cpd.5 before the activation with the profibrogenic cytokine TGFβ1 (1 ng/ml). After 48 hours of incubation, the expression of α-SMA was measured by ELISA. The obtained values were transformed into percentage inhibition over TGFβ1 control. Data are presented as mean (triplicates)±standard deviation (SD). Statistical analyses were performed by one-way ANOVA followed by Bonferroni post-hoc tests, using Sigma Plot 11.0 software. [*: p<0.05; **: p<0.01; ***: p<0.001 (comparison versus TGFμ1 1 ng/mL group)]. The curve fitting and the calculation of half maximal inhibitory concentration (IC₅₀) were performed with XLFit software 5.3.1.3.

FIG. 4: Cpd.5 inhibits TFGβ1-induced expression of α-SMA protein in human cardiac fibroblasts.

Cpd.5 was added to serum-deprived cardiac fibroblasts (NHCF) 1 hour before the activation with TGFβ1 (3 ng/ml). After 48 hours of incubation, the expression of α-SMA was measured by ELISA. The obtained values were transformed into percentage inhibition over TGFβ1 control. Data are presented as mean (triplicates)±standard deviation (SD). Statistical analyses were performed by one-way ANOVA followed by Bonferroni post-hoc tests, using Sigma Plot 11.0 software. [*: p<0.05; **: p<0.01; ***: p<0.001 (comparison versus TGFβ1 3 ng/mL group)].

FIG. 5: Cpd.5 inhibits TFGβ1-induced expression of α-SMA protein in human intestinal fibroblasts.

Cpd.5 was added to serum-deprived intestinal fibroblasts (InMyoFib) 1 hour before the activation with TGFβ1 (3 ng/ml). After 48 hours of incubation, the expression of α-SMA was measured by ELISA. The obtained values were transformed into percentage inhibition over TGFβ1 control. Data are presented as mean (triplicates)±standard deviation (SD). Statistical analyses were performed by one-way ANOVA followed by Bonferroni post-hoc tests, using Sigma Plot 11.0 software. [*: p<0.05; **: p<0.01; ***: p<0.001 (comparison versus TGFβ1 3 ng/mL group)].

FIG. 6: Cpd.5 inhibits TFGβ1-induced expression of α-SMA protein in human lung fibroblasts.

Cpd.5 was added to serum-deprived lung fibroblasts (NHLF) 1 hour before the activation with TGFβ1 (1 ng/ml). After 48 hours of incubation, the expression of α-SMA was measured by ELISA. The obtained values were transformed into percentage inhibition over TGFβ1 control. Data are presented as mean (triplicates)±standard deviation (SD). Statistical analyses were performed by one-way ANOVA followed by Bonferroni post-hoc tests, using Sigma Plot 11.0 software. [*: p<0.05; **: p<0.01; ***: p<0.001 (comparison versus TGFβ1 1 ng/mL group)].

FIG. 7: The chronic oral administration of Nitazoxanide prevents CCl4-induced levels of circulating TBA concentration.

250-275 g rats were intraperitoneally injected with olive oil (ctrl group) or with CCl4 emulsified in olive oil (CCl4:olive oil 1:2 v/v, final CCl4 concentration: 2 ml/kg) twice weekly for 3 weeks. Concomitantly, the olive oil injected group was placed on control diet while the CCl4 injected groups were placed on control diet or diet supplemented with NTZ 10 mg/kg/day or 30 mg/kg/day. After the sacrifice, circulating TBA concentration was determined. Data are presented as mean±standard deviation (SD). Statistical analyses were performed by a student t-test using Sigma Plot 11.0 software: Olive Oil vs CCl4 (#: p<0.05; ##: p<0.01; ###: p<0.001) and CCl4 vs NTZ (*: p<0.05; **: p<0.01; ***: p<0.001).

### DETAILED DESCRIPTION OF THE INVENTION

Derivatives of [2-[(5-nitro-1,3-thiazol-2-yl)carbamoyl]phenyl]ethanoate (Nitazoxanide, or NTZ) have anti-fibrotic properties in several models of fibrosis as well as anticholestatic properties, as shown in the present application. Accordingly, the present invention relates to compounds of formula (I), which are derivatives of NTZ, and to novel therapeutic uses of derivatives of NTZ. In particular, the present invention relates to a compound of formula (I) for use in the treatment of a cholestatic or fibrotic disorder. Furthermore, the invention relates to the use of a compound of formula (I) for the manufacture of a medicament useful for the treatment of a cholestatic or fibrotic disorder. The invention also relates to a pharmaceutical composition comprising a compound of formula (I). The pharmaceutical composition according to the invention is useful for treating a cholestatic or fibrotic disease.

Although the causative agents or initiating events of fibrotic disorders are quite diverse and their pathogenesis is variable, a common feature in affected tissues is the presence of large numbers of activated fibroblasts called myofibroblasts (Rosenbloom, Mendoza et al. 2013). Fibrotic stimulus such as TGFβ1 can induce differentiation of fibroblasts to myofibroblasts (Leask and Abraham 2004; Leask 2007). Myofibroblasts are metabolically and morphologically distinctive fibroblasts whose activation and proliferation play a key role during the development of the fibrotic response. Furthermore, these myofibroblasts display unique biological functions including expression of proteins involved in extracellular matrix formation such as different forms of collagen, fibronectin and other ECM proteins. The induction of α-smooth muscle actin (α-SMA) expression is a recognized hallmark of quiescent fibroblast to activated myofibroblast differentiation that can be used as a physiological read-out to evaluate the potency of the drugs that interfere with the fibrotic process. Tumor Growth β factors, and especially the Tumor Growth Factor beta 1 (TGFβ1) are recognized physiological signals that induce the phenotypic transformation of fibroblasts into profibrotic myofibroblasts that express high levels of α-SMA and high levels of extracellular matrix proteins, which are then secreted and form the fibrotic scar tissue.

Moreover, it is known that the proliferation and/or the activation of fibroblasts is responsible for the production of collagen fibers and/or is responsible for the production of the extracellular matrix (Kendall and Feghali-Bostwick 2014).

Unexpectedly, a compound of formula (I) reveal antifibrotic properties since these compounds dose-dependently reduced the level of αSMA in TGFβ-induced hepatic stellate cells and in primary fibroblasts from other organs.

The prior art does not teach that anti-fibrotic effects are directly associated to compounds of formula (I).

The present invention relates to a compound of formula (I) for treating cholestasis or fibrosis, such a compound being capable of decreasing in an unexpected manner proliferation and activation of human fibroblasts including stellate cells, the main cellular type responsible for formation of extracellular matrix and fibrosis.

The present invention relates to compounds of Formula (I):

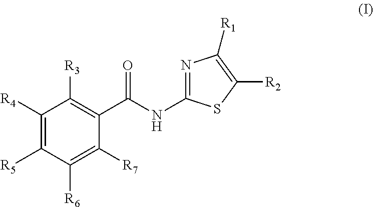

wherein

R1 represents a hydrogen atom, a deuterium atom, a halogen atom, a (C6-C14)aryl group, a heterocyclic group, a (C3-C14)cycloalkyl group, a (C1-C6)alkyl group, a sulfonyl group, a sulfoxyde group, a (C1-C6)alkylcarbonyl group, a (C1-C6)alkyloxy, a carboxylic group, a carboxylate group, a nitro group, an amino group, a (C1-C6)alkylamino group, an amido group, a (C1-C6)alkylamido group, a (C1-C6)dialkylamido group,

R2 represents a hydrogen atom, a deuterium atom, a nitro group, a (C6-C14)aryl group, a heterocyclic group, a halogen atom, a (C1-C6)alkyl group, a (C3-C14)cycloalkyl group, a (C2-C6)alkynyl group, a (C1-C6)alkyloxy group, a (C1-C6)alkylthio group, a (C1-C6)alkylcarbonyl group, a (C1-C6)alkylcarbonylamino group, a (C6-C14)arylcarbonylamino group, a carboxylic or carboxylate group, an amido group, a (C1-C6)alkylamido group, a (C1-C6)dialkylamido group, a NH2 group, a (C1-C6)alkylamino group,

or R1 and R2, together with the carbon atoms to which they are attached, form a substituted or unsubstituted 5- to 8-membered cycloalkyl, heterocyclic and aryl group,

R3, R4, R5, R6, and R7, identical or different, represent a hydrogen atom, a deuterium atom, a halogen atom, a hydroxyl group, a (C1-C6)alkylcarbonyl group, a (C1-C6)alkyl group, a (C1-C6)alkyloxy group, a (C1-C6)alkylthio group, a (C1-C6)alkylcarbonyloxy group, (C6-C14)aryloxy group, (C6-C14)aryl group, a heterocyclic group, a (C3-C14)cycloalkyl group, a nitro group, a sulfonylaminoalkyle group, a NH2 group, an amino(C1-C6)alkyl group, a (C1-C6)alkylcarbonylamino group, a carboxylic group, a carboxylate group or a R9 group;

R9 represents a O—R8 group or an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or a moiety of formula (A):

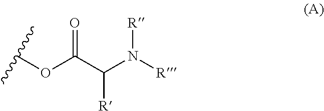

wherein R′ represents a (C1-C6)alkyl, a (C2-C6)alkenyl, a (C2-C6)alkynyl group, a (C3-C14)cycloalkyl group, a (C3-C14)cycloalkyl(C1-C6)alkyl group, cycloalkylalkenyl group, cycloalkenyl group, a cycloalkenylalkyl group, a cycloalkenylalkenyl group, a cycloalkenylalkynyl group;

R″ and R′″, independently, represent hydrogen atom, a (C1-C6)alkyl group, or a nitrogen protecting group;

R8 is a hydrogen atom, a deuterium atom, a glucuronidyl group, or a

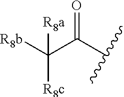

group wherein, R8a, R8b and R8c, identical or different, represent a hydrogen atom or a deuterium atom.

In a particular embodiment, the compound of the invention is of formula (I) above, with the proviso that when R2 is a nitro group (NO2) and R3 is an acetyl group (CH3CO), then R1, R3, R4, R5, R6 and R7 are not simultaneously a hydrogen atom; with the proviso that when R2 is a nitro group (NO2) and R3 is a hydroxyl group (OH), then R1, R3, R4, R5, R6 and R7 are not simultaneously a hydrogen atom.

In a particular embodiment, in the compound of formula (I) of the present invention:

an alkyl group may be a substituted or unsubstituted (C1-C6)alkyl group, in particular a substituted or unsubstituted (C1-C4)alkyl group;

an alkynyl group may be a substituted or unsubstituted (C2-C6)alkynyl group;

a cycloalkyl group may be a substituted or unsubstituted (C3-C14)cycloalkyl group;

an alkyloxy group may be a substituted or unsubstituted (C1-C6)alkyloxy group, such as a substituted or unsubstituted (C1-C4)alkyloxy group;

an alkylthio group may be a substituted or unsubstituted (C1-C6)alkylthio group, such as a substituted or unsubstituted (C1-C4)alkylthio group;

an alkylamino group may be a (C1-C6)alkylamino group, such as a (C1-C4)alkylamino group;

a dialkylamino group may be a (C1-C6)dialkylamino group, such as a (C1-C4)dialkylamino group;

an aryl group may be a substituted or unsubstituted (C6-C14)aryl group, such as a substituted or unsubstituted (C6-C14)aryl group;

a heterocyclic group may be a substituted or unsubstituted heterocycloalkyl or heteroaryl group.

In a particular embodiment, the invention relates to a compound of formula (I) wherein:

R1 represents a hydrogen atom, a deuterium atom, a halogen atom, a (C6-C14)aryl group, a heterocyclic group, a cycloalkyl group, a (C1-C6)alkyl group, a sulfonyl group, a sulfoxyde group, a (C1-C6)alkylcarbonyl group, a (C1-C6)alkyloxy, a carboxylic group, a carboxylate group, a nitro group, an amino group, a (C1-C6)alkylamino group, an amido group, a (C1-C6)alkylamido group, a dialkylamido group,

R2 represents a hydrogen atom, a deuterium atom, a nitro group, a (C6-C14)aryl group, a heterocyclic group, a halogen atom, a (C1-C6)alkyl group, a cycloalkyl group, an alkynyl group, a (C1-C6)alkyloxy group, a (C1-C6)alkylthio group, a (C1-C6)alkylcarbonyl group, a (C1-C6)alkylcarbonylamino group, a (C6-C14)arylcarbonylamino group, a carboxylic group, a carboxylate group, an amido group, a (C1-C6)alkylamido group, a dialkylamido group, an amino group, a (C1-C6)alkylamino group,

or R1 and R2, together with the carbon atoms to which they are attached, form a substituted or unsubstituted 5- to 8-membered cycloalkyl, heterocyclic and aryl group,

R4, R5, R6, and R7, identical or different, represent a hydrogen atom, a deuterium atom, a halogen atom, a hydroxyl group, a (C1-C6)alkylcarbonyl group, a (C1-C6)alkyl group, a (C1-C6)alkyloxy group, a (C1-C6)alkylthio group, a (C1-C6)alkylcarbonyloxy group, a (C6-C14)aryloxy group, a (C6-C14)aryl group, a heterocyclic group, a cycloalkyl group a nitro, a sulfonylaminoalkyle group, an amino group, an aminoalkyl group, a (C1-C6)alkylcarbonylamino group, a carboxylic group, a carboxylate group;

R3 represents a hydrogen atom, a deuterium atom, a halogen atom, a hydroxyl group, a (C1-C6)alkylcarbonyl group, a (C1-C6)alkyl group, a (C1-C6)alkyloxy group, a (C1-C6)alkylthio group, a (C1-C6)alkylcarbonyloxy group, a (C6-C14)aryloxy group, a (C6-C14)aryl group, a heterocyclic group, a cycloalkyl group a nitro, a sulfonylaminoalkyle group, an amino group, an aminoalkyl group, a (C1-C6)alkylcarbonylamino group, a carboxylic group, a carboxylate group or a R9 group;

R9 represents a O—R8 group or an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or a moiety of formula (A):

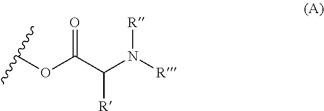

wherein R′ represents a alkyl, alkenyl, alkynyl group, cycloalkyl group, cycloalkylalkyl group, cycloalkylalkenyl group, cycloalkenyl group, a cycloalkenylalkyl group, a cycloalkenylalkenyl group, a cycloalkenylalkynyl group;

R″ and R′″, independently, represent hydrogen atom, a (C1-C6)alkyl group, or a nitrogen protecting group;

R8 is a hydrogen atom, a deuterium atom, a glucuronidyl group or a

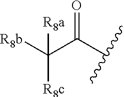

group wherein, R8a, R8b and R8c, identical or different, represent a hydrogen atom or a deuterium atom.

In a particular embodiment, R3 represents a R9 group.

In a particular embodiment, the invention relates to a compound of formula (I) wherein:

R1 represents a hydrogen atom, a halogen atom, a (C6-C14)aryl group, a heterocyclic group, a cycloalkyl group, a (C1-C6)alkyl group, a sulfonyl group, a sulfoxyde group, a (C1-C6)alkylcarbonyl group, a (C1-C6)alkyloxy, a carboxylic group, a carboxylate group, a nitro group, an amino group, a (C1-C6)alkylamino group, an amido group, a (C1-C6)alkylamido group, a dialkylamido group,

R2 represents a hydrogen atom, a nitro group, a (C6-C14)aryl group, a heterocyclic group, a halogen atom, a (C1-C6)alkyl group, a cycloalkyl group, an alkynyl group, a (C1-C6)alkyloxy group, a (C1-C6)alkylthio group, a (C1-C6)alkylcarbonyl group, a (C1-C6)alkylcarbonylamino group, a (C6-C14)arylcarbonylamino group, a carboxylic group, a carboxylate group, an amido group, a (C1-C6)alkylamido group, a dialkylamido group, an amino group, a (C1-C6)alkylamino group,

or R1 and R2, together with the carbon atoms to which they are attached, form a substituted or unsubstituted 5- to 8-membered cycloalkyl, heterocyclic and aryl group,

R3, R4, R5, R6, and R7, identical or different, represent a hydrogen atom, a halogen atom, a hydroxyl group, a (C1-C6)alkylcarbonyl group, a (C1-C6)alkyl group, a (C1-C6)alkyloxy group, a (C1-C6)alkylthio group, a (C1-C6)alkylcarbonyloxy group, a (C6-C14)aryloxy group, a (C6-C14)aryl group, a heterocyclic group, a cycloalkyl group a nitro, a sulfonylaminoalkyle group, an amino group, an aminoalkyl group, a (C1-C6)alkylcarbonylamino group, a carboxylic group, a carboxylate group;

with the proviso that when R2 is a nitro group (NO2) and R3 is an acetyl group (CH3CO), then R1, R3, R4, R5, R6 and R7 are not simultaneously a hydrogen atom,

with the proviso that when R2 is a nitro group (NO2) and R3 is a hydroxyl group (OH), then R1, R3, R4, R5, R6 and R7 are not simultaneously a hydrogen atom.

In another embodiment:

R2 represents a NO2 group;

R3 represents a O-R8 group wherein R8 represents a hydrogen atom, a deuterium

atom or a

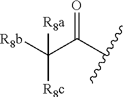

group wherein, R8a, R8b and R8c, identical or different, represent a hydrogen atom or a deuterium atom; and

R1, R4, R5, R6, and R7, identical or different, represent a hydrogen atom or a deuterium atom with the proviso that R1, R8, R8a, R8b, R8c, R4, R5, R6, and R7 are not simultaneously a hydrogen atom.

In a particular embodiment, at least one of R1, R8a, R8b and R8c, R4, R5, R6, and R7 represent a deuterium atom.

In a particular embodiment, R1, R8a, R8b and R8c, R4, R5, R6, and R7 represent a deuterium atom.

In a particular embodiment, R8a, R8b and R8c, R4, R5, R6, and R7 represent a deuterium atom.

In a particular embodiment, R1, R4, R5, R6, and R7 represent a deuterium atom.

In a particular embodiment, R3 represents a O—R8 group wherein R8 represents a

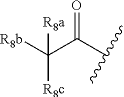

group wherein R8a, R8b and R8c represent a deuterium atom.

In a particular embodiment, R8 represents a

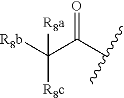

group wherein R8a, and R8b represent a deuterium atom, R8c represents a hydrogen atom. R1, R4, R5, R6, and R7 represent a hydrogen atom.

In a particular embodiment, R8 represents a

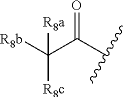

group wherein at R8a, represents a deuterium atom, R8b and R8c represent a hydrogen atom, R1, R4, R5, R6, and R6 represent a hydrogen atom.

In another particular embodiment:

R2 represents a NO2 group;

R1, R4, R5, R6 and R7 represent a hydrogen atom; and

R3 is a O—R8 group wherein R8 represents a

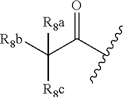

group wherein, R8a, R8b and R8c, identical or different, represent a hydrogen atom or a deuterium atom, with the proviso that R8a, R8b, R8c are not simultaneously a hydrogen atom.

In a particular embodiment, at least one R8a, R8b and R8c represent a deuterium atom.

In a further particular embodiment, R8a, R8b and R8c represent a deuterium atom.

In another embodiment, R8a and R8b represent a deuterium atom, R8c represents a hydrogen atom.

In yet another embodiment, R8a represents a deuterium atom, R8b and R8c represent a hydrogen atom.

In a further particular embodiment, the compound of formula (I) is selected from:
2-[(5-nitro-1,3-thiazol-2-yl)carbamoyl]phenyl (d3)ethanoate;
2-[(5-nitro-1,3-thiazol-2-yl)carbamoyl]phenyl (d2) ethanoate; and
2-[(5-nitro-1,3-thiazol-2-yl)carbamoyl]phenyl (d1) ethanoate.

In another embodiment, the compound of formula (I) is selected from:

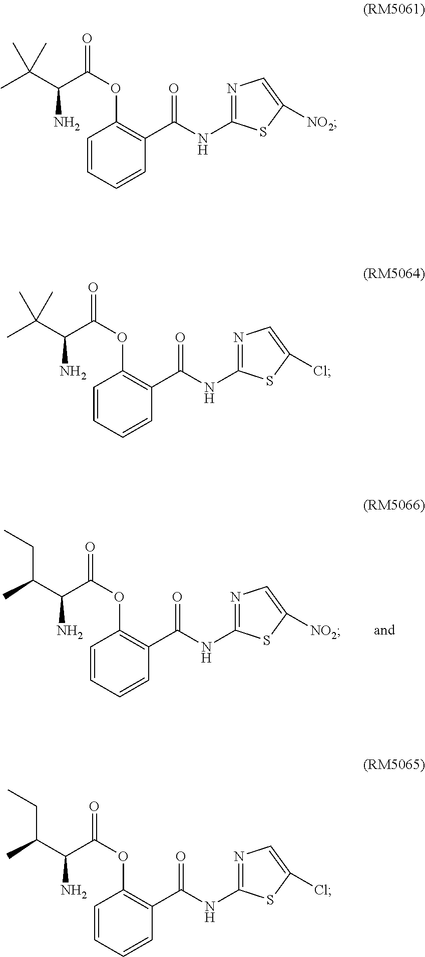 [SMILES: CC(C)(C)[C@H](N)C(=O)Oc1ccccc1C(=O)Nc1ncc([N+](=O)[O-])s1 ; CC(C)(C)[C@H](N)C(=O)Oc1ccccc1C(=O)Nc1ncc(Cl)s1 ; CC[C@H](C)[C@H](N)C(=O)Oc1ccccc1C(=O)Nc1ncc([N+](=O)[O-])s1 ; CC[C@H](C)[C@H](N)C(=O)Oc1ccccc1C(=O)Nc1ncc(Cl)s1]

or a pharmaceutically acceptable salt thereof, such as a hydrochloride salt thereof.

The term “alkyl” refers to a saturated hydrocarbon radical that is linear or branched, substituted or not, having preferably from one to six, and even more preferably from one to four carbon atoms, such as methyl, ethyl, n-propyl, isopropyl, n-butyl, isobutyl, tert-butyl, or sec-butyl. The alkyl group can be optionally substituted by one or more halogen atoms, by an aryl group or by a heterocyclic group or by a cycloalkyl group. Further possible substituents of an alkyl group also include one or more substituents selected from an amino group, an alkylamino group, a dialkylamino group, an oxime group, a hydroxyl group, an alkyloxy group, an aryloxy group, a carboxylic group, a carboxylate, an amido group, an oxime, alkenyl group and an alkynyl group.

The term alkynyl denotes linear or branched hydrocarbon groups containing from 2 to 6 carbon atoms and containing at least one triple bond. Examples of alkynyl containing from 3 to 6 carbon atoms are 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the isomeric forms thereof. The alkyl group can be optionally substituted for example by an amino group or a hydroxyl group.

The terms “alkyloxy” and “alkylthio” refer to an alkyl group as defined above that is linked to the remainder of the compound by an oxygen or sulfur atom, respectively.

The term “aryloxy” refers to an aryl group as defined above that is linked to the remainder of the compound by an oxygen atom.

The term “alkylamino” refers to monoalkylamino (—NHR) or dialkylamino (—NRR′) group where R and R′ independently represent an alkyl group as defined above. In a particular embodiment, the alkyl group(s) of the alkylamino group may be substituted or not with a cycloalkyl group, an aryl group, a heterocyclic group, or an alkyloxycarbonyl group.

The term “cycloalkylamino” refers to a —NH-cycloalkyl group or a —N(alkyl)cycloalkyl group.

The term “amino group” designates a —NH2 group.

The term “nitro group” refers to a —NO2 group.

The term “hydroxyl group” refers to a —OH group.

The term “sulfonyl group” refers to a —SO2 group.

The term “sulfoxyde group” refers to a —SO group.

The term “oxime group” refers to a C═N—OH group.

The term “alkylamido” refers to monoalkylamido (—CONHR) or dialkylamido (—CONRR′) group where R and R′ independently represent an alkyl group as defined above.

The term “amido group” refers to a —CONH2 group.

The term “cycloalkyl” designates a substituted or unsubstituted alkyl group that forms one cycle having preferably from three to fourteen carbon atoms, and more preferably three to six carbon atoms, such as cyclopropyl, cyclopentyl and cyclohexyl. The cycloalkyl group of the present invention may be unsubstituted, or substituted, for example with an alkyl group, in particular with an alkyl group substituted with one or more halogen atoms, such as the CF3 group.

The term “carbonyl” designates a —CO group.

The term “aryl” designates an aromatic group, substituted or not, having preferably from six to fourteen carbon atoms such as phenyl, a-naphtyl, b-naphtyl, or biphenyl.

The term “heterocyclic” refers to a heterocycloalkyl group or a heteroaryl group. The term “heterocycloalkyl” group refers to a cycloalkyl as indicated above, substituted or not, that further comprises one or several heteroatoms selected among nitrogen, oxygen or sulfur. They generally comprise from four to fourteen carbon atoms, such as morpholinyl, piperazinyl, piperidinyl, pyrrolidinyl, tetrahydropyranyl, dithiolanyl and azepanyl groups. In a particular embodiment, the heterocycloalkyl group is a 5-, 6- or 7-membered cycle. The term “heteroaryl” refers to an aryl group as indicated above, substituted or not, that further comprises one or several heteroatoms selected among nitrogen, oxygen or sulfur. They generally comprise from four to fourteen carbon atoms. In a particular embodiment, the heteroaryl group is a 5-, 6- or 10-membered cycle. Representative heteroaryl groups include a pyridinyl, pyrimidinyl, furanyl, thiophenyl, quinoleinyl, and isoquinoleinyl group.

The aryl group or the heterocyclic group can be optionally substituted by one or more halogen atom(s), alkyl group(s), or alkyloxy group(s).

By halogen atom, an atom of bromine, chlorine, fluorine or iodine is understood, in particular an atom of bromine, chlorine or fluorine.

Specific compounds according to the invention include:
Cpd.1: 2′-(benzo[d]thiazol-2-ylcarbamoyl)-[1,1′-biphenyl]-2-carboxylic acid;
Cpd.2: N-(5-benzamido-4-(thiophen-2-yl)-1,3-thiazol-2-yl)-2-methoxybenzamide;
Cpd.3: 2,6-difluoro-N-(5-methyl-4-phenyl-1,3-thiazol-2-yl)benzamide;
Cpd.4: 2-chloro-N-[4-(2-naphthyl)-1,3-thiazol-2-yl]-5-nitrobenzamide;
Cpd.5: 2-[(5-nitro-1,3-thiazol-2-yl)carbamoyl]phenyl (d3)ethanoate;
Cpd.6: 2-[(5-nitro-1,3-thiazol-2-yl)carbamoyl]phenyl (d2) ethanoate;
Cpd.7: 2-[(5-nitro-1,3-thiazol-2-yl)carbamoyl]phenyl (d1) ethanoate;
Cpd.8: 2-(5-nitrothiazol-2-ylcarbamoyl)phenyl 2-amino-3,3-dimethylbutanoate;
Cpd.9: 2-(5-chlorothiazol-2-ylcarbamoyl)phenyl 2-amino-3,3-dimethylbutanoate;
Cpd.10: 2-(5-nitrothiazol-2-ylcarbamoyl)phenyl 2-amino-3-methylpentanoate;
Cpd.11: 2-(5-chlorothiazol-2-ylcarbamoyl)phenyl 2-amino-3-methylpentanoate;
Cpd.12: RM5061 ((S)-2-(5-nitrothiazol-2-ylcarbamoyl)phenyl 2-amino-3,3-dimethylbutanoate);
Cpd.13: RM5064 ((S)-2-(5-chlorothiazol-2-ylcarbamoyl)phenyl 2-amino-3,3-dimethylbutanoate);
Cpd. 14: RM5066 ((2S,3S)-2-(5-nitrothiazol-2-ylcarbamoyl)phenyl 2-amino-3-methylpentanoate); and
Cpd.15: RM5065 ((2S,3S)-2-(5-chlorothiazol-2-ylcarbamoyl)phenyl 2-amino-3-methylpentanoate).

In a particular embodiment, the compound of formula (I) is compound 12.

According to the present invention, the terms “fibrosis”, “fibrotic disease”, “fibrotic disorder” and declinations thereof denote a pathological condition of excessive deposition of fibrous connective tissue in an organ or tissue. More specifically, fibrosis is a pathological process, which includes a persistent fibrotic scar formation and overproduction of extracellular matrix by the connective tissue, as a response to tissue damage. Physiologically, the deposit of connective tissue can obliterate the architecture and function of the underlying organ or tissue.

According to the present invention, the fibrosis or fibrotic disorder may be associated with any organ or tissue fibrosis. Illustrative, non-limiting examples of particular organ fibrosis include liver, gut, kidney, skin, epidermis, endodermis, muscle, tendon, cartilage, heart, pancreas, lung, uterus, nervous system, testis, penis, ovary, adrenal gland, artery, vein, colon, intestine (e.g. small intestine), biliary tract, soft tissue (e.g. mediastinum or retroperitoneum), bone marrow, joint or stomach fibrosis, in particular liver, kidney, skin, epidermis, endodermis, muscle, tendon, cartilage, heart, pancreas, lung, uterus, nervous system, testis, ovary, adrenal gland, artery, vein, colon, intestine (e.g. small intestine), biliary tract, soft tissue (e.g. mediastinum or retroperitoneum), bone marrow, joint, eye or stomach fibrosis.

According to the present invention, the terms “cholestasis” or “cholestatic disease”, or “cholestatic disorder” and declinations thereof denote a pathological condition defined by a decrease in bile flow due to impaired secretion by hepatocytes or to obstruction of bile flow through intra- or extrahepatic bile ducts. Therefore, the clinical definition of cholestasis is any condition in which substances normally excreted into bile are retained.

In a particular embodiment, the fibrotic disorder is selected in the group consisting of a liver, gut, lung, heart, kidney, muscle, skin, soft tissue (e.g. mediastinum or retroperitoneum), bone marrow, intestinal, and joint (e.g. knee, shoulder or other joints) fibrosis.

In a preferred embodiment, the fibrotic disorder is selected in the group consisting of liver, lung, skin, kidney and intestinal fibrosis.

In a more preferred embodiment of the present invention, treated fibrotic disorder is selected in the group consisting of the following non exhaustive list of fibrotic disorders: non-alcoholic steatohepatitis (NASH), pulmonary fibrosis, idiopathic pulmonary fibrosis, skin fibrosis, eye fibrosis (such as capsular fibrosis), endomyocardial fibrosis, mediastinal fibrosis, myelofibrosis, retroperitoneal fibrosis, progressive massive fibrosis (a complication of coal workers' pneumoconiosis), proliferative fibrosis, neoplastic fibrosis, lung fibrosis consecutive to chronic inflammatory airway disease (COPD, asthma, emphysema, smoker's lung, tuberculosis), alcohol or drug-induced liver fibrosis, liver cirrhosis, infection-induced liver fibrosis, radiation or chemotherapeutic-induced fibrosis, nephrogenic systemic fibrosis, Crohn's disease, ulcerative colitis, keloid, old myocardial infarction, scleroderma/systemic sclerosis, arthrofibrosis, some forms of adhesive capsulitis, chronic fibrosing cholangiopathies such as Primary Sclerosing Cholangitis (PSC) and Primary Biliary Cholangitis (PBC), biliary atresia, familial intrahepatic cholestasis type 3 (PFIC3), peri-implantational fibrosis and asbestosis.

According to a particular embodiment of the invention, the cholestatic disease is selected in the group consisting of primary biliary cholangitis (PBC), primary sclerosing cholangitis (PSC), Intrahepatic Cholestasis of Pregnancy, Progressive Familial Intrahepatic Cholestasis, Biliary atresia, Cholelithiasis, Infectious Cholangitis, Cholangitis associated with Langerhans cell histiocytosis, Alagille syndrome, Nonsyndromic ductal paucity, Drug-induced cholestasis, and Total parenteral nutrition-associated cholestasis. In a preferred embodiment, the cholestatic disease is PBC or PSC, in particular PBC.

The term “treatment” or “treating” refers to the curative or preventive treatment of a cholestatic or fibrotic disorder in a subject in need thereof. The treatment involves the administration of the compound, in particular comprised in a pharmaceutical composition, to a subject having a declared disorder, i.e. to a patient, to cure, delay, reverse, or slow down the progression of the disorder, improving thereby the condition of the subject. A treatment may also be administered to a subject that is healthy or at risk of developing a cholestatic or fibrotic disorder to prevent or delay the disorder.

Therefore, according to the invention, the treatment of a cholestatic or fibrotic disorder involves the administration of a compound of formula (I), or of a pharmaceutical composition containing the same, to a subject having a declared disorder to cure, delay, reverse or slow down the progression of the disorder, thus improving the condition of the patient or to a healthy subject, in particular a subject who is at risk of developing a cholestatic or fibrotic disorder.

The subject to be treated is a mammal, preferably a human. The subject to be treated according to the invention can be selected on the basis of several criteria associated with cholestatic or fibrotic diseases such as previous drug treatments, associated pathologies, genotype, exposure to risk factors, viral infection, as well as on the basis of the detection of any relevant biomarker that can be evaluated by means of imaging methods and immunological, biochemical, enzymatic, chemical, or nucleic acid detection methods.

In a particular embodiment, the treatment of a fibrotic disorder may comprise the administration of a combination of two or more compounds of formula (I). According to a variant of this embodiment, the two or more compounds of formula (I) are comprised in a single composition. In another variant of this embodiment, the two or more compounds of formula (I) are for simultaneous, sequential or separate administration in therapy, therefore being possibly included in different compositions.

In case of sequential administration, a compound of formula (I) may be administered prior to the administration of another.

Compounds of formula (I) can be formulated as pharmaceutically acceptable salts particularly acid or base salts compatible with pharmaceutical use. Salts of compounds of formula (I) include pharmaceutically acceptable acid addition salts, pharmaceutically acceptable base addition salts, pharmaceutically acceptable metal salts, ammonium and alkylated ammonium salts. These salts can be obtained during the final purification step of the compound or by incorporating the salt into the previously purified compound. The invention further relates to a pharmaceutical composition comprising a compound of formula (I), in a pharmaceutically acceptable carrier.

In a preferred embodiment, the present invention concerns a pharmaceutical composition comprising a compound selected from compounds 1 to 15 shown above, for use in a method of treatment of a cholestatic or fibrotic disease.

The pharmaceutical compositions comprising a compound of formula (I) can also comprise one or several excipients or vehicles, acceptable within a pharmaceutical context (e.g. saline solutions, physiological solutions, isotonic solutions, etc., compatible with pharmaceutical usage and well-known by one of ordinary skill in the art).

These compositions can also further comprise one or several agents or vehicles chosen among dispersants, solubilisers, stabilisers, preservatives, etc. Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc.

These compositions can be formulated in the form of injectable suspensions, syrups, gels, oils, ointments, pills, tablets, suppositories, powders, gel caps, capsules, aerosols, etc., eventually by means of galenic forms or devices assuring a prolonged and/or slow release. For this kind of formulations, agents such as cellulose, carbonates or starches can advantageously be used. The compositions of the present invention can also be formulated in the form of liposome delivery systems, such as small unilamellar vesicles, large unilamellar vesicles, and multilamellar vesicles. Liposomes can be formed from a variety of lipids, including but not limited to amphipathic lipids such as phosphatidylcholines, sphingomyelins, phophatidylcholines, cardiolipins, phosphatidylethanolamines, phosphatidylserines, phosphatidylglycerols, phosphatidic acids, phosphatidylinositols, diacyl trimethylammonium propanes, diacyl dimethylammonium propanes, and stearylamine, neutral lipids such as triglycerides, and combinations thereof. The pharmaceutical combination of the Compounds of Formula (I) can be administered in different ways and in different forms. Thus, for example, it can be administered in a systematic way, or parenteral way, by using oral, topical, perlingual, nasal, rectal, transmucosal, transdermal, intestinal, intramuscular, intravenously, subcutaneous, intraarterial, intraperitoneal, intrapulmonary or intraocular route, by using methods known in the art.

Formulations for oral administration may be in the form of aqueous solutions and suspensions, in addition to solid tablets and capsule formulations. The aqueous solutions and suspensions may be prepared from sterile powders or granules. The compounds may be dissolved in water, polyethylene glycol, propylene glycol, ethanol, corn oil, cottonseed oil, peanut oil, sesame oil, benzyl alcohol, sodium chloride, and/or various buffers.

Compounds of formula (I) may be administered by different routes and in different forms. For example, the derivative(s) may be administered via a systemic way, per os, parenterally, by inhalation, by nasal spray, by nasal instillation, or by injection, such as for example intravenously, by intra-muscular route, by subcutaneous route, by transdermal route, by topical route, by intra-arterial route, etc.

Of course, the route of administration will be adapted to the form of the compound of formula (I) according to procedures well known by those skilled in the art.

Compounds of formula (I) are administered in a therapeutically effective amount. Within the context of the invention, the term “effective amount” refers to an amount of the compound sufficient to produce the desired therapeutic result.

The frequency and/or dose relative to the administration can be adapted by one of ordinary skill in the art, in function of the patient, the pathology, the form of administration, etc. Typically, compounds of formula (I) can be administered for the treatment of a fibrotic disease at a dose comprised between 0.01 mg/day to 4000 mg/day, such as from 50 mg/day to 2000 mg/day, and particularly from 100 mg/day to 1000 mg/day. Administration can be performed daily or even several times per day, if necessary. In one embodiment, the compound is administered at least once a day, such as once a day, twice a day, or three times a day. In a particular embodiment, the compound is administered once or twice a day. In particular, oral administration may be performed once a day, during a meal, for example during breakfast, lunch or dinner, by taking a tablet comprising the compound at a dose of about 1000 mg, in particular at a dose of 1000 mg. In another embodiment, a tablet is orally administered twice a day, such as by administering a first tablet comprising the compound at a dose of about 500 mg (i.e. at a dose of 450 to 550 mg), in particular at a dose of 500 mg, during one meal, and administering a second tablet comprising the compound at a dose of about 500 mg, in particular at a dose of 500 mg, during another meal the same day. Suitably, the course of treatment with a compound of formula (I) is for at least 1 week, in particular for at least 2, 3, 4, 5, 6, 7, 8, 9, 10, 15, 20 or 24 weeks or more. In particular, the course of treatment with a compound of formula (I) is for at least 1 year, 2 years, 3 years, 4 years or at least 5 years.

In a particular embodiment, the invention relates to the combination of a compound of formula (I) with at least one statin (HMG-CoA reductase inhibitors) such as pravastatin, fluvastatin, atorvastatin, lovastatin, simvastatin, rosuvastatin, mevastatin, cerivastatin, and pitavastatin. Statins may be in the form of a salt, hydrate, solvate, polymorph, or a co-crystal. Statins may also be in the form of a hydrate, solvate, polymorph, or a co-crystal of a salt. Statins may also be present in the free acid of lactone form according to the present invention.

In a particular embodiment, the invention relates to the use of a compound of formula (I) for the treatment of a cholestatic or fibrotic disease, in combination with at least one other therapeutically active agent with known antifibrotic activity. According to a variant of this embodiment, the compound of formula (I) can be combined with any antifibrotic compound such as pirfenidone or receptor tyrosine kinase inhibitors (RTKIs) such as Nintedanib, Sorafenib and other RTKIs, or angiotensin II (AT1) receptor blockers, or CTGF inhibitor, or any antifibrotic compound susceptible to interfere with the TGFβ- and BMP-activated pathways including activators of the latent TGFβ complex such as MMP2, MMP9, THBS1 or cell-surface integrins, TGFβ receptors type I (TGFBRI) or type II (TGFBRII) and their ligands such as TGFβ, Activin, inhibin, Nodal, anti-Müllerian hormone, GDFs or BMPs, auxiliary co-receptors (also known as type III receptors), or components of the SMAD-dependent canonical pathway including regulatory or inhibitory SMAD proteins, or members of the SMAD-independent or non-canonical pathways including various branches of MAPK signaling, TAK1, Rho-like GTPase signaling pathways, phosphatidylinositol-3 kinase/AKT pathways, TGFβ-induced EMT process, or canonical and non-canonical Hedgehog signaling pathways including Hh ligands or target genes, or any members of the WNT, or Notch pathways which are susceptible to influence TGFβ signaling.

Thus, the invention also relates to a pharmaceutical composition comprising a compound of formula (I), or a pharmaceutically acceptable salt of a compound of formula (I), in combination with at least one therapeutically active agent with known antifibrotic activity selected from pirfenidone or receptor tyrosine kinase inhibitors (RTKIs) such as Nintedanib, Sorafenib and other RTKIs, or angiotensin II (AT1) receptor blockers, or CTGF inhibitor, or antifibrotic compound susceptible to interfere with the TGFβ- and BMP-activated pathways including activators of the latent TGFβ complex such as MMP2, MMP9, THBS1 or cell-surface integrins, TGFβ receptors type I (TGFBRI) or type II (TGFBRII) and their ligands such as TGFβ, Activin, inhibin, Nodal, anti-Müllerian hormone, GDFs or BMPs, auxiliary co-receptors (also known as type III receptors), or components of the SMAD-dependent canonical pathway including regulatory or inhibitory SMAD proteins, or members of the SMAD-independent or non-canonical pathways including various branches of MAPK signaling, TAK1, Rho-like GTPase signaling pathways, phosphatidylinositol-3 kinase/AKT pathways, TGFβ-induced EMT process, or canoninal and non-canonical Hedgehog signaling pathways including Hh ligands or target genes, or any members of the WNT, or Notch pathways which are susceptible to influence TGFβ signaling, for use in a method for treating a fibrotic disorder.

In another particular embodiment, other classes of molecules that could also be combined with a compound of formula (I) include statins (HMG-CoA reductase inhibitors) such as pravastatin, fluvastatin, atorvastatin, lovastatin, simvastatin, rosuvastatin mevastatin, cerivastatin, and pitavastatin, JAK/STAT inhibitors, or other anti-inflammatory and/or immunosuppressant agents. The non-exhaustive list of these agents includes but is not limited to glucocorticoids, NSAIDS, cyclophosphamide, nitrosoureas, folic acid analogs, purine analogs, pyrimidine analogs, methotrexate, azathioprine, mercaptopurine, ciclosporin, myriocin, tacrolimus, sirolimus, mycophenolic acid derivatives, fingolimod and other sphingosine-1-phosphate receptor modulators, monoclonal and/or polyclonal antibodies against such targets as proinflammatory cytokines and proinflammatory cytokine receptors, T-cell receptor, integrins. Other classes of molecules that could also be combined with compounds of formula (I) include molecules that could potentially enhance the exposure or the effect of compounds of formula (I).

In another embodiment, a compound of formula (I), or a combination of compounds of formula (I) is administered as the sole active ingredient. Accordingly, the invention also relates to a pharmaceutical composition comprising a compound selected from a compound of formula (I), or a pharmaceutically acceptable salt of a compound of formula (I), for use in a method for treating a cholestatic or fibrotic disorder, wherein said compound(s) is(are) the only active ingredient(s) in the composition.

In a further embodiment, the present invention provides methods of treating a cholestatic or fibrotic disease comprising the administration of one or more compounds of formula (I), in particular in the form of a pharmaceutical composition containing one or more compounds of formula (I).

Synthesis of compounds of formula (I) may be carried to methods known in the art, such as those mentioned below and in WO2016/077420.

Specific reaction intermediates can be synthesized and purified from compounds that may be already available commercially or that can readily be synthesized.

General scheme of synthesis of the compounds of General Formula (I) is presented in the following schemes:

Desired N-(thiazol-2-yl)benzamide derivatives can be obtained from appropriate benzoic acid/chloride and appropriate aminothiazole intermediates according to methods well known by a person skilled in the art.

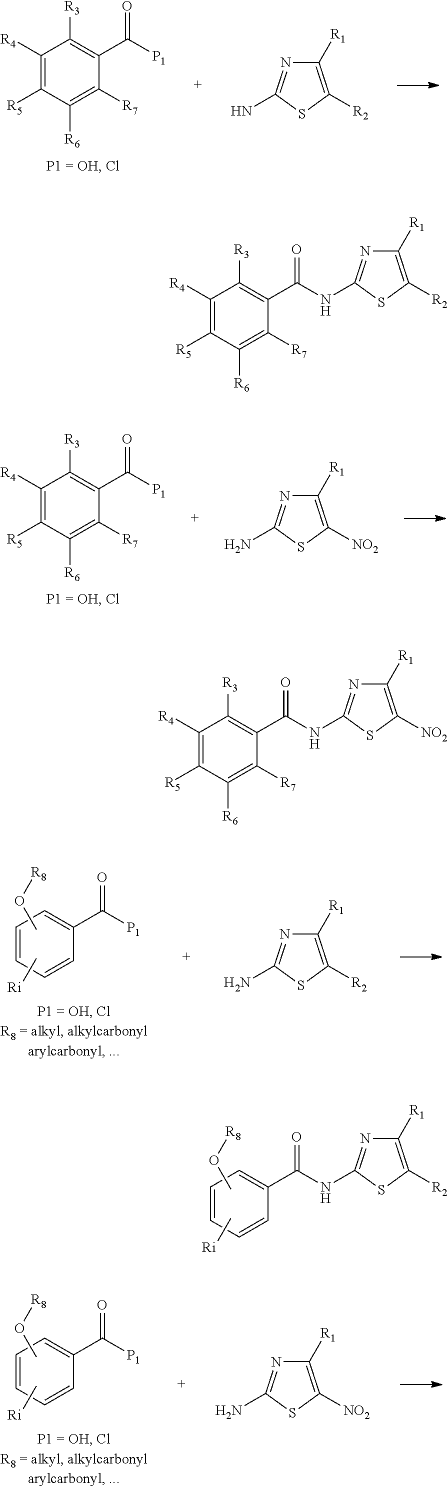

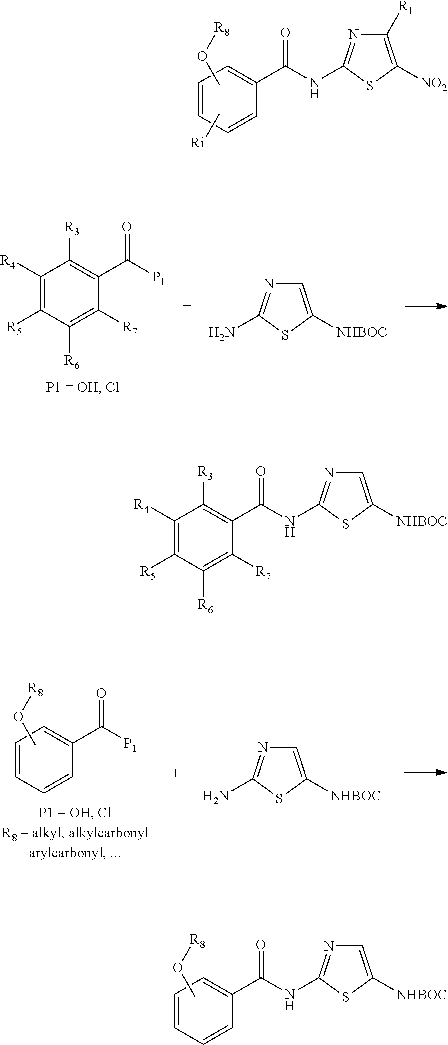

In another aspect of the invention, further functionalization may be introduced once the N-(thiazol-2-yl)benzamide scaffold is formed. For example, hydroxy benzamide derivatives may be further substituted by an alkyl or an alkylcarbonyl group as shown in the scheme below; other halogeno benzamide derivatives may undergo palladium catalyzed coupling reactions to introduce the desired aryle, heteroaryle cycles that may be commercially available or synthesized by the man skilled in the art. Still in another aspect of the invention a wide range of 5N-(5-aminothiazol-2-yl)benzamide derivatives can be prepared by deprotection and further functionalization of tert-butyl 2-(benzamido)thiazol-5-ylcarbamate.

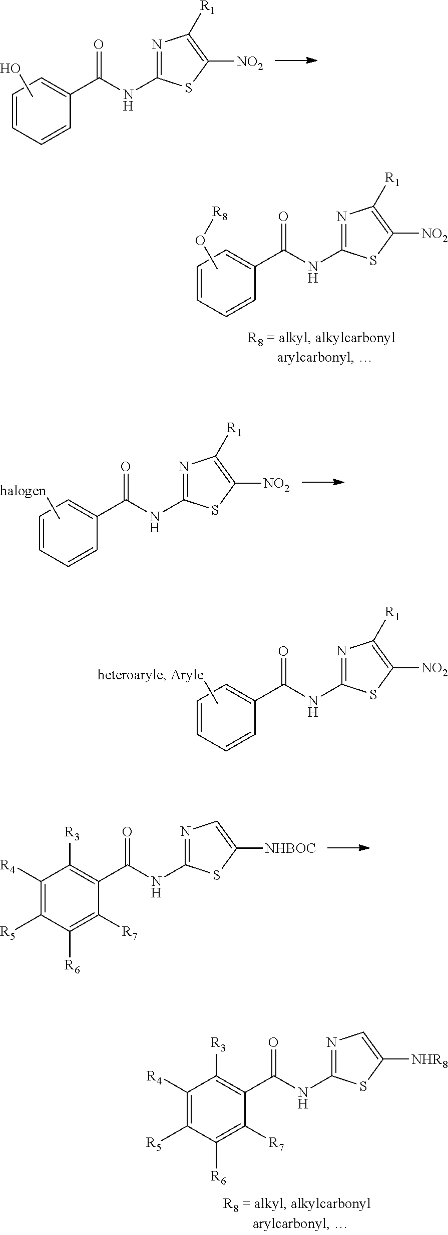

Specific thiazole intermediates include 5-nitro-1,3-thiazol-2-amine (commercially available from Combi-Blocks, cat #HI-1112) or 5-nitro-4-substituted-1,3-thiazol-2-amines such as for example 4-methyl-5-nitro-1,3-thiazol-2-amine and 5-nitro-4-phenyl-1,3-thiazol-2-amine that are commercially available or can be synthesized according to the methods described respectively by (Tokumitsu and Hayashi 1985) or (Singh, Singh et al. 2003). Other 4-substituted 5-nitro-1,3-thiazol-2-amines such as 5-nitro-4-aryl-1,3-thiazol-2-amine, 5-nitro-4-trifluoromethyl-1,3-thiazol-2-amine are accessible by using the same methods as described by (Kikelj and Urleb 2002) and (Tasaganva, Tambe et al. 2011), while 4-methylamino-5-nitro-1,3-thiazol-2-amine derivatives can be synthesized from 5-nitro-2-acetamido-4-formylthiazole, which synthesis was described by (Silberg, Frenkel et al. 1963).

Other specific thiazoles can be purchased from usual providers such as for examples, 2-amino-5-phenylthiazole (Combi-Blocks, cat #ST-4301), 2-amino-4-phenylthiazole (Combi-Blocks, cat #HC-2218), 5-ethyl-1,3-thiazol-2-amine (Combi-Blocks, cat #QC-6305), 4-ethyl-1,3-thiazol-2-amine (Combi-Blocks, cat #H1-1797), 4-methyl-1,3-thiazol-2-amine (Combi-Blocks, cat #H1-1202), 2-amino-4-(2-pyridyl)thiazole (Alfa Aesar, cat #H58554), 2-amino-4-(3-pyridyl)thiazole (Alfa Aesar, cat #H58630), 5-ethyl-1,3-thiazol-2-amine (Ark Pharm, cat #AK132917). More generally, specific thiazole intermediates can be synthesized by using the methods well-known by one of ordinary skill in the art such as the reaction of the appropriate thiourea with appropriate α-haloketones or related compounds. Further specific thiazoles can be obtained by using techniques well known by a person skilled in the art so as to get 4 or 5-substituted aryl, heteroarylthiazole derivatives. Similarly 5-nitrothiazoles may be reduced to their amino analogs by usual techniques such as palladium catalyzed hydrogenation (Pevarello, Amici et al. 2004) or iron catalyzed reduction (Funahashi, Tsuruoka et al. 2007).

The functional groups optionally present in the reaction intermediates that are generated for obtaining the desired compounds of formula (I) can be protected, either permanently, or temporarily, by protective groups, which ensure unequivocal synthesis of the desired compounds. The reactions of protection and deprotection are carried out according to techniques well known by a person skilled in the art or such as those described in the literature, as in the book “Greene's Protective Groups in Organic Synthesis” (Wuts and Greene 2007).

The compounds according to the invention may contain one or more asymmetric centers. The present invention includes stereoisomers (diastereoisomers, enantiomers), pure or mixed, as well as racemic mixtures and geometric isomers, or tautomers of compounds of formula (I). When an enantiomerically pure (or enriched) mixture is desired, it can be obtained either by purification of the final product or of chiral intermediates, or by asymmetric synthesis according to methods known by a person skilled in the art (using for example chiral reactants and catalysts). Certain compounds according to the invention can have various stable tautomeric forms and all these forms and mixtures thereof are included in the invention. The techniques for obtaining and characterizing the stereoisomers, pure or mixed, as well as racemic mixtures and geometric isomers, or tautomers are described in the literature, such as in the book “Chirality in Drug Design and Development” (Reddy and Mehvar 2004).

The details of the general methods of synthesis and purification of intermediate and final reaction products for compounds of formula (I) containing one or more deuterium atom(s) are provided in the examples. Specific reaction intermediates can be synthesized and purified from compounds that may be already available commercially or that can readily be synthesized. For example deuterated acetyl chloride can be commercially available or synthetized from corresponding carboxylic acids (said carboxylic acids can be synthetized according to the method described by (Ginsburg and Hescheles, 1958))

General scheme of synthesis of the compounds of Formula (I) is presented in the following scheme:

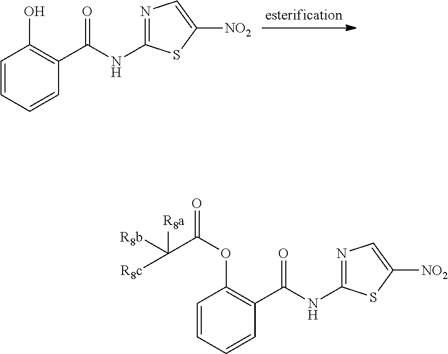

The esterification step is carried out according to techniques well known by a person skilled in the art.

The compounds of Formula (I) can be purified by precipitation or solid/liquid extraction after evaporation of the reaction medium. Further or other purification step can be performed by chromatography over silica gel or by crystallization, when the compound is stable as a solid form, by applying techniques well known in the literature or, more in general, for chemicals (Armarego and Chai 2009).

Moreover, the required purification and/or (re-)crystallization steps that are appropriate for isolating compounds of formula (I) from the reaction mixture, can be used for obtaining amorphous, polymorphous, mono- or poly-crystalline forms. Such polymorphisms may present distinct pharmacological and/or chemical properties, for example in terms of solubility, intrinsic dissolution rate, melting temperature, bioavailability, and/or possible transition from a polymorphic state to another one in pharmaceutical compositions and/or biological fluids.

The (re-)crystallisation assays can be performed in panels of different solvents (such as isopropanol, acetone, methanol, diisopropyl ether or water) or mixture thereof, and by applying different conditions, such as reaction volumes or temperatures. The resulting samples can be analyzed by different techniques such as microscopy, calorimetry, and/or spectroscopy that allow establishing the features of a particular crystalline form, such as structure, solubility, stability or conversion to other forms (Bauer 2004; Morissette, Almarsson et al. 2004; Erdemir, Lee et al. 2007; Yin and Grosso 2008). Such a polymorphism study allows characterizing the crystalline form of a compound that is pharmaceutically acceptable for both pharmacological and manufacturing points of view.

Certain compounds of formula (I) can be isolated in the form of zwitterions and each of these forms is included in the invention, as well as mixtures thereof.

Specific compounds of formula (I) can comprise at least one atom of the structure that is replaced by an isotope (radioactive or not). Examples of isotopes that can be included in the structure of the compounds according to the invention can be selected from hydrogen, carbon, nitrogen, oxygen, sulphur such as 2H, 3H, 13C, 14C, 15N, 18O, 17O, 35S respectively. When non-radioactive, the stable isotope can be selectively incorporated in the structure in place of hydrogen (in the case of deuterium) or carbon (in the case of 13C) not only as means of performing absorption, distribution, metabolism, and excretion (ADME) studies but also as means for obtaining compounds that may retain the desired biochemical potency and selectivity of the original compound while the metabolic fate is substantially altered. In some favourable cases, this modification has the potential to have a positive impact effect on safety, efficacy and/or tolerability of the original compound (Mutlib 2008). Otherwise radioactive isotopes 3H and 14C are particularly preferred as they are easy to prepare and detect in studies of the bioavailability in vivo of the substances. The heavy isotopes (such as 2H) are particularly preferred as they are used as internal standards in analytical studies and as possible variants of pharmaceutical interest.

The phrase “pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability.

The term “carrier”, “vehicle”, or “excipient” refers to any substance, not itself a therapeutic agent, that is added to a pharmaceutical composition to be used as a carrier, vehicle, and/or diluent for the delivery of a therapeutic agent to a subject in order to improve its handling or storage properties or to permit or facilitate formation of a dosage unit of the composition into a discrete article. The pharmaceutical compositions of the invention, either individually or in combination, can comprise one or several agents or vehicles chosen among dispersants, solubilisers, stabilisers, preservatives, etc.

The invention is further described with reference to the following, non-limiting, examples.

### EXAMPLES

### Materials and Methods

### Synthesis of a Deuterated Compound According to the Invention

Chemical names follow IUPAC nomenclature. Starting materials and solvents are purchased from commercial suppliers (Acros Organic, Sigma Aldrich, Combi-Blocks, Fluorochem, Fluka, Alfa Aesar or Lancaster) and are used as received without further purification. Some starting materials can be readily synthesized by a person skilled in the art. Air and moisture sensitive reactions are carried out under an inert atmosphere of nitrogen, and glassware was oven-dried. No attempts are made to optimize reaction yields. Thin-layer chromatography (TLC) is done on Merck silica gel 60 UV254 (250 μm) plates. Visualization is accomplished with UV light. Column chromatography is performed on Geduran silica gel 60 (40-63 μm) from Merck. Melting points (mp) are recorded with a Büchi Melting Point B-545 and are uncorrected. All microwave irradiation experiments are carried out in a Biotage Initiator microwave apparatus. 1H spectra were recorded on Bruker Advance I spectrometer at 300 MHz. Chemical shifts (δ) are reported in ppm (parts per million), by reference to the hydrogenated residues of deuterated solvent as internal standard: 2.50 ppm for DMSO-d6, 7.26 ppm for CDCI3, and 3.31, and 4.78 for Methanol-d4. The spectral splitting patterns are designated as follows: s, singlet; d, doublet; dd, doublet of doublets; ddd, doublet of doublet of doublets; t, triplet; dt, doublet of triplets; q, quartet; m, multiplet; br s, broad singlet. Coupling constants (J) are quoted to the nearest 0.1 Hz. All tested compounds exhibited 95% chemical purity assessed by HPLC on a Merck HITACHI Lachrom L-7000 series and Merck HITACHI diode array detector L-7455 with a Waters column Symmetry C18 (3.5 μm, 4.6*75 mm) and using a gradient of MeOH/Millipore water containing 0.1% of formic acid. Mass spectrometry measurements were performed on qTOF Waters Micromass Ultima API and AutoPurification System 2767 with an Acquity QDa detector from Waters. All solvents are HPLC grade.

### Example 1

Cpd.5: 2-[(5-nitro-1,3-thiazol-2-yl)carbamoyl]phenyl (d3)acetate;

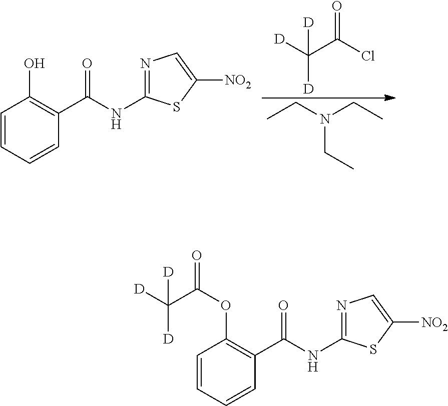

Triethylamine (0.112 mL, 0.829 mmol) and (²H)₃ acetyl chloride (0.031 mL, 0.434 mmol) was added to a mixture of 2-hydroxy-N-(5-nitro-1,3-thiazol-2-yl)benzamide (Interchim réf:RP253/Batch: WZG110215-OA02) (0.1 g, 0.377 mmol) in dichloromethane (0.38 M). The reaction was stirred at room temperature for 2 hours. The mixture was diluted with water and extracted twice with ethyl acetate. Combined organic layers were washed with brine, dried over MgSO4, filtered and concentrated under reduced pressure. The crude product was purified on silica gel (dichloromethane/ethyl acetate: 9/1) to afford 2-[(5-nitro-1,3-thiazol-2-yl)carbamoyl]phenyl (²H₃)acetate as a yellow solid (0.038 g, 0.122 mmol, yield: 28%). NMR 1H (300 MHz, DMSO-d6, δ ppm): 7.31 (dd, 1H, J=8.1 Hz, J=0.9 Hz, HAr), 7.41-7.47 (m, 1H, HAr), 7.65-7.71 (m, 1H, HAr), 7.84 (dd, 1H, J=7.8 Hz, J=1.5 Hz, HAr), 8.69 (s, 1H, HAr), 13.61 (br(s), 1H, NH) ; Mass (ESI+): 311.2 (M+H)+, 333.2 (M+Na)+, 643.3 (2M+Na)+, 659.3 (2M+K)+; MP: 197-200° C.

### In Vitro Evaluation of the Compounds According to the Invention

Compounds were dissolved in dimethyl sulfoxide (DMSO, Fluka cat #41640).

TABLE 1
Examples of compounds of formula (I)

| Compound | Structure |
|---|---|
| Cpd. 1 | 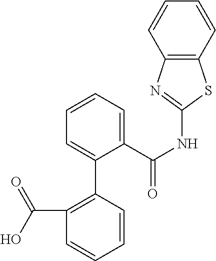 [SMILES: O=C(O)c1ccccc1-c1ccccc1C(=O)Nc1nc2ccccc2s1] |
| Cpd. 2 | 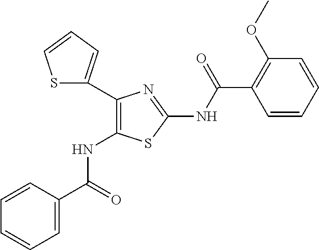 [SMILES: COc1ccccc1C(=O)Nc1nc(-c2cccs2)c(NC(=O)c2ccccc2)s1] |
| Cpd. 3 | 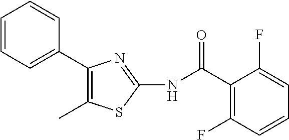 [SMILES: Cc1sc(NC(=O)c2c(F)cccc2F)nc1-c1ccccc1] |
| Cpd. 4 | 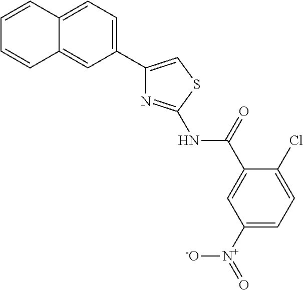 |

RM-5061 was synthesized according to the method described in the application WO2016/077420.
   hHSC Culture

The human primary hepatic stellate cells (hHSC) (Innoprot) were cultured in STeCM medium (ScienCell cat #5301) that was supplemented with 2% fetal bovine serum (FBS, ScienCell cat #0010), 1% penicillin/streptomycin (ScienCell cat #0503) and stellate cell growth supplement (SteCGS; ScienCell cat #5352). Cell culture flasks were coated with Poly-L Lysine (Sigma cat #P4707) for a better adherence.

Activation of hHSC with TGF-β1

The human primary hepatic stellate cells (hHSC) (Innoprot) were cultured under standard conditions, as described above. The cells were subsequently plated at a density of 2×10⁴cells/well or 6,5×10³ cells/well into 96-well or 384-well plates respectively. The next day, cell-culture medium was removed, and cells were washed with PBS (Invitrogen cat #14190). Then, hHSC were deprived for 24 hours in serum-free and SteCGS-free medium. For the treatments with compounds of formula (I), the serum-deprived hHSC were preincubated for 1 hour with the compounds followed by addition of the profibrogenic stimuli TGFβ1 (PeproTech cat #100-21, 1 ng/mL) in serum-free and SteCGS-free medium for an additional 48 hour period. At the end of treatment, the cell culture supernatants of treated-HSC were transferred into a new plate before storage at −20° C.The cells were washed with PBS (Invitrogen, cat #14190) before the addition of lysis buffer (CelLytic™ MT reagent; Sigma #C3228). Plates were then incubated for 30 min on ice using a plate shaker, before storage at −20° C.

Activation of NHCF-V with TGFβ1

The Normal Human Cardiac Fibroblasts (ventricle) (NHCF-V) (Lonza) were isolated from normal, adult heart tissue. Cells were cultured in Fibroblast Basal Medium (FBM) (Lonza cat #CC-3131) that was supplemented with FGM™-3 BulletKit™ kit (Lonza cat #CC-4525). The complete medium contains 10% fetal bovine serum. For the activation experiments with TGFβ1, the NHCF-V were plated at a density of 6×10³ cells per well in 96-well plates. The next day, cell-culture medium was removed, and cells were washed with PBS (Invitrogen cat #14190). NHCF were deprived for 24 hours in serum-free, insulin-free and rhFGF-B-free medium. For the treatments with deuterated compounds of formula (I), the serum-deprived NHCF were preincubated for 1 hour with the compounds followed by addition of the profibrogenic stimulus TGFβ1 (PeproTech cat #100-21, 3 ng/mL) in serum-free insulin-free and rhFGF-B-free medium for an additional 48 hour period.

Activation of InMyoFib with TGβ1

The Human Intestinal Myofibroblast (InMyoFib) (Lonza) were cultured in Smooth Muscle Cell Basal Medium (SmBM-2 TM) (Lonza cat #CC-3181) that was supplemented with SmGMTM-2 BulletKit TM (Lonza cat #CC-4149). The complete medium contains 5% fetal bovine serum. For the activation experiments with TGFβ1, the inMyoFib were plated at a density of 10×10³ cells per well in 96-well plates. The next day, cell-culture medium was removed, and cells were washed with PBS (Invitrogen cat #14190). InMyoFib were deprived for 24 hours in serum-free, insulin-free and rhFGF-B-free medium. For the treatments with deuterated compounds of formula (I), the serum-deprived InMyoFib were preincubated for 1 hour with the compounds followed by addition of the profibrogenic stimuli TGFβ1 (PeproTech cat #100-21, 3 ng/mL) in serum-free insulin-free and rhFGF-B-free medium for an additional 48 hour period.

Activation of NHLF with TGβ1

The Normal Human Lung Fibroblasts (NHLF) (Lonza) were cultured in Fibroblast Basal Medium (FBM) (Lonza cat #CC-3131) that was supplemented with FGM-2 SingleQuots™ Kit (Lonza cat #CC-3132). The complete medium contains 2% fetal bovine serum. For the activation experiments with TGFβ1, the NHLF were plated at a density of 5×10³ cells per well in 96-well plates. The next day, cell-culture medium was removed, and cells were washed with PBS (Invitrogen cat #14190). NHLF were deprived for 24 hours in serum-free, insulin-free and rhFGF-B-free medium. For the treatments with compounds of formula (I), the serum-deprived NHLF were preincubated for 1 hour with the compounds followed by addition of the profibrogenic stimuli TGFβ1 (PeproTech cat #100-21, 1 ng/mL) in serum-free, insulin-free and rhFGF-B-free medium for an additional 48 hour period. At the end of treatment, cells were washed with PBS (Invitrogen, cat #14190) before the addition of lysis buffer (CelLyticTM MT reagent; Sigma #C 3228). Plates were then incubated for 30 min on ice using a plate shaker, before storage at −20° C.

### α-SMA ELISA

The level of α-SMA was measured using a Sandwich ELISA. Briefly, the wells of an ELISA plate were first coated with the capture antibody (mouse monoclonal anti-ACTA2, Abnova) at 4° C. overnight. After 3 washes in PBS +0.2% Tween 20, a blocking solution consisting of PBS +0.2% BSA was added for one hour followed by another washing cycle. The cell lysates were transferred into the wells for binding to the capture antibody for a period of 2 h at room temperature. After the washing procedure, the detection antibody (biotinylated mouse monoclonal anti-ACTA2, Abnova) was added for 2 hours at room temperature followed by 3 washes. For the detection, an HRP-conjugated Streptavidin (R&D Systems cat #DY998) was first applied for 30 min at room temperature. After washing, the HRP substrate TMB (;BD, #555214) was added and incubated for 7 min at room temperature in the dark. Upon oxidation, TMB forms a water-soluble blue reaction product that becomes yellow with addition of sulfuric acid (solution stop), enabling accurate measurement of the intensity at 450 nm using a spectrophotometer. The developed color is directly proportional to the amount of α-SMA present in the lysate.

### Col1α1 ELISA

The level of the human pro-Collagen α1 (col1α1) was measured using a Sandwich ELISA (R&D systems,). Briefly, the wells of an ELISA plate were first coated with the capture antibody at room temperature overnight. After 3 washes in PBS +0.05% Tween 20, a blocking solution consisting of PBS +1% BSA was added for one hour followed by another washing cycle. The cell culture supernatants of treated-HSC were transferred into the wells for binding to the capture antibody for a period of 2 h at room temperature. After the washing procedure, the biotinylated detection antibody was added for 2 hours at room temperature followed by 3 washes. For the detection, an HRP-conjugated Streptavidin was first applied for 20 min at room temperature. After washing, the HRP substrate TMB (BD, cat #555214) was added and incubated for 20 min at room temperature in the dark. Upon oxidation, TMB forms a water-soluble blue reaction product that becomes yellow with addition of sulfuric acid (solution stop), enabling accurate measurement of the intensity at 450 nm using a spectrophotometer. The developed color is directly proportional to the amount of the collal protein present in the cell culture supernatant.

In vivo Evaluation of the Compounds According to the Invention

### Evaluation of RM-5061 in a Chronic CCl4-Induced Liver Fibrosis Model

9 week-old C57BL/6 mice will be placed on control diet or diet supplemented with RM-5061 for 6 weeks. 3 regimen containing RM-5061 will be prepared corresponding respectively to an exposure of NTZ 10, 30, or 100 mg/kg/day. Concomitantly, and for the total duration of 6 weeks, the mice will be treated 3 times a week with CCl4 dissolved in olive oil or vehicle by oral gavage. The amount of CCl4 will be progressively increased from 0.875 ml/kg to 2.5 ml/kg. The last day of treatment, the mice will be sacrificed after a 6 h fasting period. Blood samples will collected for biochemical analyses of serum. The liver will be rapidly excised for biochemical, histological and expression studies

### Evaluation of RM-5061 in a Chronic CDAAc Diet-Induced Liver Fibrosis Model

The antifibrotic effect of NTZ will be assessed in a murine model of CDAAc diet-induced experimental liver fibrosis. 6 week-old C57BL/6 mice will be fed for 12 weeks a control (CSAA) diet, CDAAc diet, or CDAAc diet supplemented with RM-5061 10,30,100 mg/kg/day for 12 weeks.

The body weight and the food intake will be monitored twice per week. On the last day of treatment, mice will be sacrificed after a 6 h fasting period. The liver will be rapidly excised for biochemical and histological studies.

### Evaluation of the Impact of NTZ on Plasmatic Bile Acid Concentration

OFA Sprague Dawley rats (initial body weight 250-275 g) were randomized according to their body weight into 4 groups and treated for 3 weeks. The rats were intraperitoneally injected with olive oil (ctrl group) or with CCl4 emulsified in olive oil (CCl4:olive oil 1:2 v/v, final CCl4 concentration: 2 ml/kg) twice weekly. Concomitantly, the olive oil injected group was placed on control diet while the CCl4 injected groups were placed on control diet or diet supplemented with NTZ. 2 regimen containing NTZ were prepared corresponding respectively to an exposure of 10 or 30 mg/kg/day. The last day of treatment, the rats were sacrificed after a 6 h fasting period. Blood samples were collected and the serum was isolated for biochemical analyses.

### Measurement of Plasmatic Concentration of Total Bile Acids

The plasmatic concentration of Total Bile Acids (TBA) was determined using the appropriate Randox kit for the Daytona automated analyzer (Randox, cat #BI 3863). In the presence of Thio-NAD, the enzyme 3-α hydroxysteroid dehydrogenase (3-α HSD) converts bile acids to 3-keto steroids and Thio-NADH. The reaction is reversible and 3-α HSD can convert 3-ketosteroids and Thio-NADFH-to bile acids and Thio-NAD. In the presence of excess NADH, the enzyme cycling occurs efficiently and the rate of formation of Thio-NADH is determined by measuring specific change of absorbance at 405 nm. Results are expressed in μmol/L.

### Results and Conclusions

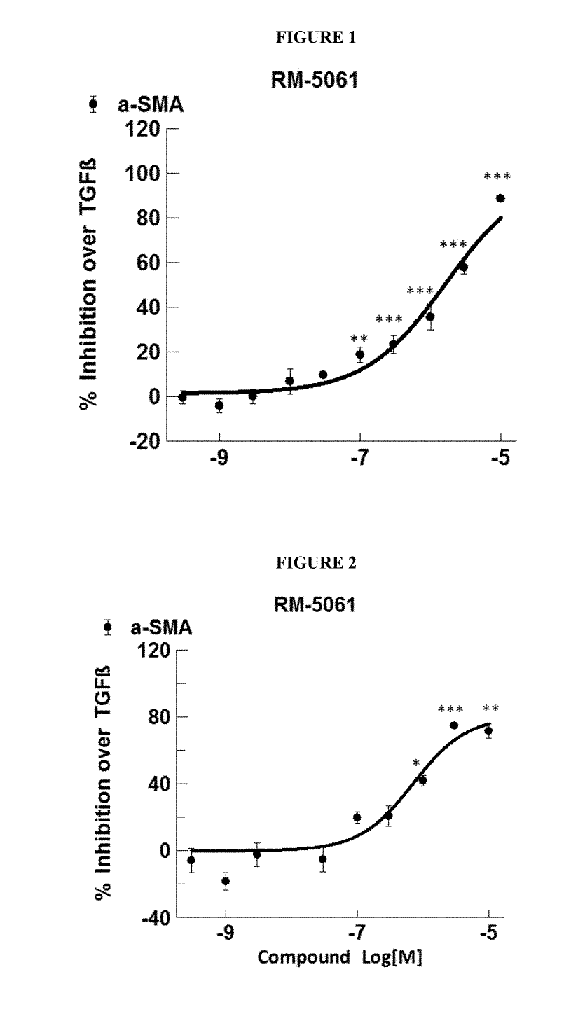

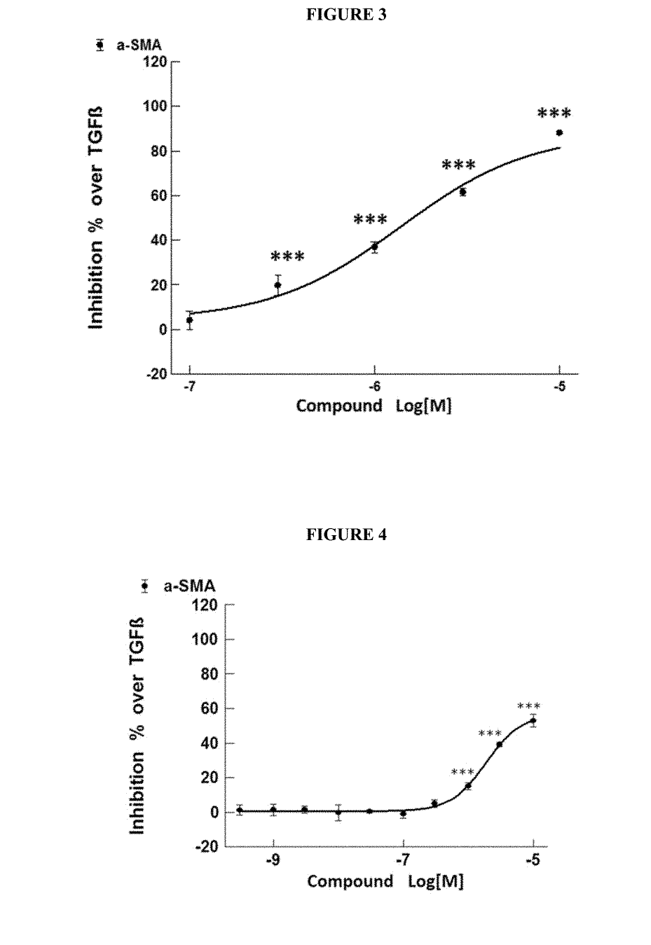

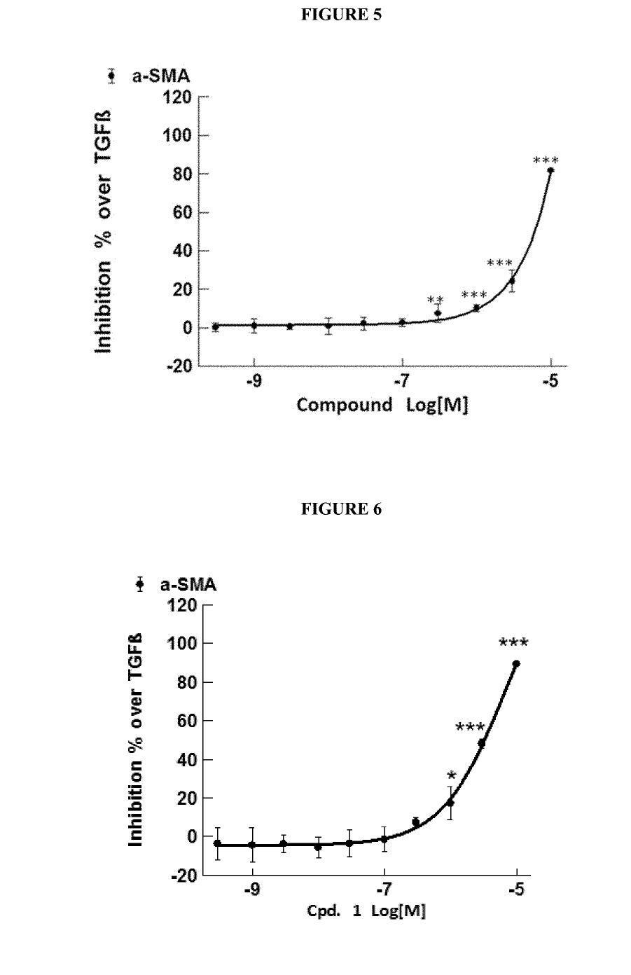

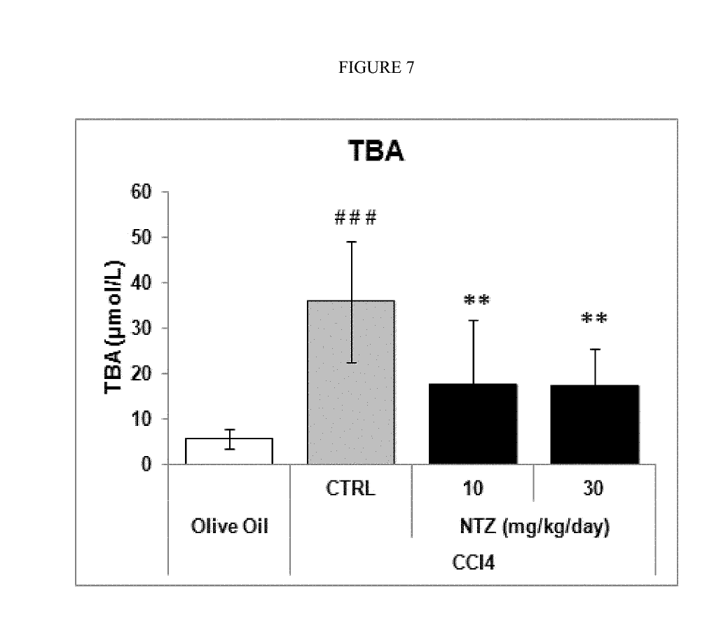

The abnormal persistence of differentiated myofibroblasts is a characteristic of many fibrotic diseases. Following liver injury, quiescent HSC undergo a process of activation that is characterized by a differentiation into (α-SMA)-positive myofibroblasts. In an attempt to find new antifibrotic molecules, compounds of Formula (I) and deuterated derivative compounds of formula (I) were phenotypically screened in a model of human HSC activated with the profibrogenic cytokine TGFβ1. The levels of α-SMA, a hallmark of fibrotic lesions, were used to evaluate the potency of the compounds to interfere with the fibrotic process. Several Compounds of Formula (I) revealed antifibrotic properties as illustrated in Table 2. For example, RM-5061 dose-dependently reduced the level of α-SMA (FIG. 1. Table 3) with an IC₅₀ comprised between 0.1 and 3 μM. These antifibrotic properties were also observed in NHLF (FIG. 2) with a comparable IC₅₀ Moreover, another marker of TGFβ stimulation was reduced in a comparable manner by RM-5061 such as the extracellular matrix collagen 1A1 (COL1A1) (Table 3).

Deuterated derivative compounds of formula (I) reduced in a dose-response manner α-SMA levels in TGFβ-activated HSC with an IC₅₀ comprised between 0.1 and 3 μM(FIG. 3). In addition, the antifibrotic potential of deuterated derivative compounds of formula (I) was extended to fibroblasts derived from other tissues, including normal human cardiac fibroblasts (FIG. 4). human Intestinal myofibroblasts (InMyoFib) (FIG. 5) and normal human lung fibroblasts (NHLF) (FIG. 6),In all these models of fibrosis, deuterated derivative compounds of formula showed significant antifibrotic effects at a concentration of 1 μM.

Toxicity assays confirmed that the reduced levels of α-SMA and COL1A1 were not due to toxicity or apoptosis of cells (data not shown).

Altogether, these results suggest that compounds of formula (I) and deuterated derivative compounds of formula (I) can provide therapeutic benefits in multiple types of fibrotic diseases.

Moreover, in the in vivo model of CCl4-induced liver injury, NTZ prevented the pathological increase of circulating total bile acid concentration (FIG. 7), which is a marker associated with cholestasis. Therefore, the applicant has discovered unexpected anticholestatic activities for the antiparasitic agent NTZ. It is expected that the compounds of formula (I) disclosed herein, which are NTZ derivatives, have also anticholestatic properties, in particular compounds 5 to 11, which are also prodrugs of TZ, the active metabolite of NTZ.

Table 2: Nitazoxanide derivatives inhibit TGFβ1-induced expression of α-SMA protein in human HSC.

Serum-deprived HSC were preincubated for 1 hour with NTZ derivatives at 3 μM before the activation with the profibrogenic cytokine TGFβ1 (1 ng/ml). After 48 hours of incubation, the expression of α-SMA was measured by ELISA. The obtained values were transformed into percentage inhibition over TGFβ1 control. Data are presented as mean of quadruplicates.

| | % I over TGFβ [Cpd] = 3μM |
|---|---|
| Cpd1 | 40% |
| Cpd2 | 43% |
| Cpd3 | 48% |
| Cpd4 | 53% |

Table 3: RM-5061 inhibits TGFβ1-induced expression of α-SMA & COL1A1 proteins in human HSC.

Serum-deprived HSC were preincubated for 1 hour with RM-5061 before the activation with the profibrogenic cytokine TGFβ1 (1 ng/ml). After 48 hours of incubation, the cellular α-SMA content as well as secreted COL1A1 were measured by ELISA. The obtained values were transformed into percentage inhibition over TGFβ1 control. Data are presented as mean (quadruplicates). The curve fitting and the calculation of half maximal inhibitory concentration (IC₅₀) were performed with XLFit software 5.3.1.3.

| | IC50 (μM) | Max % I Over TGFβ |
|---|---|---|
| αSMA | 2.79 | 40% |
| COL1A1 | 2.67 | 43% |

### REFERENCES

Armarego, W. L. F. and C. L. L. Chai (2009). Purification of Laboratory Chemicals (Sixth Edition), ELSEVIER.
Bauer, M. (2004). Polymorphisme et stabilité. Paris, FRANCE, Editions de santé.
de Carvalho, L. P. S., C. M. Darby, et al. (2011). “Nitazoxanide disrupts membrane potential and intrabacterial pH homeostasis of Mycobacterium tuberculosis.” ACS Med. Chem. Lett. 2(Copyright (C) 2015 American Chemical Society (ACS). All Rights Reserved.): 849-854.
Di Santo, N. and J. Ehrisman (2014). “A functional perspective of nitazoxanide as a potential anticancer drug.” Mutat. Res., Fundam. Mol. Mech. Mutagen. 768(Copyright (C) 2015 American Chemical Society (ACS). All Rights Reserved.): 16-21.
Dubreuil, L., I. Houcke, et al. (1996). “In vitro evaluation of activities of nitazoxanide and tizoxanide against anaerobes and aerobic organisms.” Antimicrob. Agents Chemother. 40(Copyright (C) 2015 American Chemical Society (ACS). All Rights Reserved.): 2266-2270.
Erdemir, D., A. Y. Lee, et al. (2007). “Polymorph selection: the role of nucleation, crystal growth and molecular modeling.” Curr Opin Drug Discov Devel 10(6): 746-755.
Finegold, S. M., D. Molitoris, et al. (2009). “Study of the in vitro activities of rifaximin and comparator agents against 536 anaerobic intestinal bacteria from the perspective of potential utility in pathology involving bowel flora.” Antimicrob. Agents Chemother. 53(Copyright (C) 2015 American Chemical Society (ACS). All Rights Reserved.): 281-286.
Fox, L. M. and L. D. Saravolatz (2005). “Nitazoxanide: a new thiazolide antiparasitic agent.” Clin. Infect. Dis. 40(Copyright (C) 2015 American Chemical Society (ACS). All Rights Reserved.): 1173-1180.
Funahashi, Y., A. Tsuruoka, et al. (2007). Preparation of urea derivatives containing nitrogenous aromatic ring compounds as inhibitors of angiogenesis, Eisai Co., Ltd, Japan . 458 pp., Cont.-in-part of Appl. No. PCT/JP401/09221.
Hemphill, A., J. Mueller, et al. (2006). “Nitazoxanide, a broad-spectrum thiazolide anti-infective agent for the treatment of gastrointestinal infections.” Expert Opin. Pharmacother. 7(Copyright (C) 2015 American Chemical Society (ACS). All Rights Reserved.): 953-964.
Hoffman, P. S., G. Sisson, et al. (2007). “Antiparasitic drug nitazoxanide inhibits the pyruvate oxidoreductases of Helicobacter pylori, selected anaerobic bacteria and parasites, and Campylobacter jejuni.” Antimicrob. Agents Chemother. 51(Copyright (C) 2015 American Chemical Society (ACS). All Rights Reserved.): 868-876.
Kendall, R. T. and C. A. Feghali-Bostwick (2014). “Fibroblasts in fibrosis: novel roles and mediators.” Front Pharmacol 5: 123.
Kikelj, D. and U. Urleb (2002). “Product class 17: thiazoles.” Sci. Synth. 11(Copyright (C) 2016 American Chemical Society (ACS). All Rights Reserved.): 627-833.
Leask, A. (2007). “TGFbeta, cardiac fibroblasts, and the fibrotic response.” Cardiovasc Res 74(2): 207-212.
Leask, A. and D. J. Abraham (2004). “TGF-beta signaling and the fibrotic response.” FASEB J 18(7): 816-827.
Megraudd, F., A. Occhialini, et al. (1998). “Nitazoxanide, a potential drug for eradication of Helicobacter pylori with no cross-resistance to metronidazole.” Antimicrob. Agents Chemother. 42(Copyright (C) 2015 American Chemical Society (ACS). All Rights Reserved.): 2836-2840.
Morissette, S. L., O. Almarsson, et al. (2004). “High-throughput crystallization: polymorphs, salts, co-crystals and solvates of pharmaceutical solids.” Adv Drug Deliv Rev 56(3): 275-300.
Mutlib, A. E. (2008). “Application of stable isotope-labeled compounds in metabolism and in metabolism-mediated toxicity studies.” Chem Res Toxicol 21(9): 1672-1689.
Pankuch, G. A. and P. C. Appelbaum (2006). “Activities of tizoxanide and nitazoxanide compared to those of five other thiazolides and three other agents against anaerobic species.” Antimicrob. Agents Chemother. 50 (Copyright (C) 2015 American Chemical Society (ACS). All Rights Reserved.): 1112-1117.
Pevarello, P., R. Amici, et al. (2004). Preparation of arylmethyl-carbonylamino-thiazole derivatives for treating cell proliferative disorders with an altered cell dependent kinase activity, Pharmacia Italia S.P.A., Italy . 21 pp., Cont.-in-part of U.S. Ser. No. 372,832.
Reddy, I. K. and R. Mehvar (2004). Chirality in Drug Design and Development, CRC Press.
Rosenbloom, J., F. A. Mendoza, et al. (2013). “Strategies for anti-fibrotic therapies.” Biochim Biophys Acta 1832(7): 1088-1103.
Rossignol, J.-F. (2014). “Nitazoxanide: A first-in-class broad-spectrum antiviral agent.” Antiviral Res. 110(Copyright (C) 2015 American Chemical Society (ACS). All Rights Reserved.): 94-103.
Rossignol, J. F. and R. Cavier (1975). 2-Benzamido-5-nitrothiazoles, S. P. R. L. Phavic, Belg. . 11 pp.
Rossignol, J. F. and H. Maisonneuve (1984). “Nitazoxanide in the treatment of Taenia saginata and Hymenolepis nana infections.” Am J Trop Med Hyg 33(Copyright (C) 2015 U.S. National Library of Medicine.): 511-512.
Silberg, A., Z. Frenkel, et al. (1963). “Thiazoles. V. The preparation and properties of 5-nitro-2-acetamido-4-formylthiazole.” Chem. Ber. 96(Copyright (C) 2016 American Chemical Society (ACS). All Rights Reserved.): 2992-2995.
Singh, K., S. Singh, et al. (2003). “Monoazo disperse dyes. Part 3: synthesis and fastness properties of some novel 4,5-disubstituted thiazolyl-2-azo disperse dyes.” Color. Technol. 119(Copyright (C) 2016 American Chemical Society (ACS). All Rights Reserved.): 198-204.
Tasaganva, R. G., S. M. Tambe, et al. (2011). “Synthesis and characterization of thermally stable second-order nonlinear optical side-chain polyurethanes containing nitro-substituted oxadiazole and thiazole chromophores.” J. Mol. Struct. 1000(Copyright (C) 2016 American Chemical Society (ACS). All Rights Reserved.): 10-23.
Tokumitsu, T. and T. Hayashi (1985). “Reaction of β-nitroenamines with electrophilic reagents. Synthesis of β-substituted β-nitroenamines and 2-imino-5-nitro-4-thiazolines.” J. Org. Chem. 50(Copyright (C) 2016 American Chemical Society (ACS). All Rights Reserved.): 1547-1550.
Wuts, P. G. M. and T. W. Greene (2007). Greene's Protective Groups in Organic Synthesis, Fourth Edition, John Wiley & Sons.
Yin, S. X. and J. A. Grosso (2008). “Selecting and controlling API crystal form for pharmaceutical development—strategies and processes.” Curr Opin Drug Discov Devel 11(6): 771-777.

We claim:

1. A method of treating a cholestatic or fibrotic disorder comprising administering a compound of formula (I), or a pharmaceutically acceptable salt thereof, to a subject having a cholestatic or fibrotic disorder: 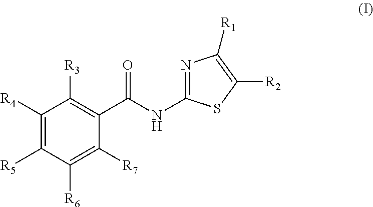
wherein
R1 represents a hydrogen atom, a deuterium atom, a halogen atom, a (C6-C14)aryl group, a heterocyclic group, a (C3-C14)cycloalkyl group, a (C1-C6)alkyl group, a sulfonyl group, a sulfoxyde group, a (C1-C6)alkylcarbonyl group, a (C1-C6)alkyloxy, a carboxylic group, a carboxylate group, a nitro group, an amino group, a (C1-C6)alkylamino group, an amido group, a (C1-C6)alkylamido group, a (C1-C6)dialkylamido group;
R2 represents a hydrogen atom, a deuterium atom, a nitro group, a (C6-C14)aryl group, a heterocyclic group, a halogen atom, a (C1-C6)alkyl group, a (C3-C14)cycloalkyl group, a (C2-C6)alkynyl group, a (C1-C6)alkyloxy group, a (C1-C6)alkylthio group, a (C1-C6)alkylcarbonyl group, a (C1-C6)alkylcarbonylamino group, a (C6-C14)arylcarbonylamino group, a carboxylic or carboxylate group, an amido group, a (C1-C6)alkylamido group, a (C1-C6)dialkylamido group, a NH2 group, a (C1-C6)alkylamino group;
or R1 and R2, together with the carbon atoms to which they are attached, form a substituted or unsubstituted 5- to 8-membered cycloalkyl, heterocyclic and aryl group;
R3, R4, R5, R6, and R7, identical or different, represent a hydrogen atom, a deuterium atom, a halogen atom, a hydroxyl group, a (C1-C6)alkylcarbonyl group, a (C1-C6)alkyl group, a (C1-C6)alkyloxy group, a (C1-C6)alkylthio group, a (C1-C6)alkylcarbonyloxy group, a (C6-C14)aryloxy group, a (C6-C14)aryl group, a heterocyclic group, a (C3-C14)cycloalkyl group, a nitro group, a sulfonylaminoalkyle group, a NH2 group, an amino(C1-C6)alkyl group, a (C1-C6)alkylcarbonylamino group, a carboxylic group, a carboxylate group, or a R9 group;
R9 represents a O—R8 group or an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or a moiety of formula (A): 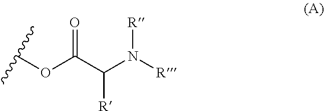
wherein R′ represents a alkyl, alkenyl, alkynyl group, cycloalkyl group, cycloalkylalkyl group, cycloalkylalkenyl group, cycloalkenyl group, a cycloalkenylalkyl group, a cycloalkenylalkenyl group, a cycloalkenylalkynyl group;
R″ and R′″, independently, represent hydrogen atom, an alkyl group, or a nitrogen protecting group;
R8 is a hydrogen atom, a deuterium atom, a glucuronidyl group, or a 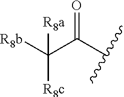
group wherein, R8a, R8b and R8c, identical or different, represent a hydrogen atom or a deuterium atom;
with the proviso that compound of formula (I) is not NTZ or TZ.

2. The method according to claim 1, wherein:
R2 represents a NO2 group;
R3 represents a O—R8 group wherein R8 represents a hydrogen atom, a deuterium atom or a 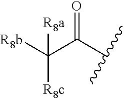
group wherein, R8a, R8b and R8c, identical or different, represent a hydrogen atom or a deuterium atom; and
R1, R4, R5, R6, and R7, identical or different, represent a hydrogen atom or a deuterium atom with the proviso that R1, R8, R8a, R8b, R8c, R4, R5, R6, and R7 are not simultaneously a hydrogen atom.

3. The method according to claim 1, wherein said compound is selected from the group consisting of:
2′-(benzo[d]thiazol-2-ylcarbamoyl)[1,1′-biphenyl]-2-carboxylic acid;
N-(5-benzamido-4-(thiophen-2-yl)-1,3-thiazol-2-yl)-2-methoxybenzamide;
2,6-difluoro-N-(5-methyl-4-phenyl-1,3-thiazol-2-yl)benzamide;
2-chloro-N-[4-(2-naphthyl)-1,3-thiazol-2-yl]-5-nitrobenzamide;
2-[(5-nitro-1,3-thiazol-2-yl)carbamoyl]phenyl (d3)ethanoate;
2-[(5-nitro-1,3-thiazol-2-yl)carbamoyl]phenyl (d2) ethanoate;
2-[(5-nitro-1,3-thiazol-2-yl)carbamoyl]phenyl (d1) ethanoate;
2-(5-nitrothiazol-2-ylcarbamoyl)phenyl 2-amino-3,3-dimethylbutanoate;
2-(5-chlorothiazol-2-ylcarbamoyl)phenyl 2-amino-3,3-dimethylbutanoate;
2-(5-nitrothiazol-2-ylcarbamoyl)phenyl 2-amino-3-methylpentanoate;
2-(5-chlorothiazol-2-ylcarbamoyl)phenyl 2-ami no-3-methyl pentanoate;
RM5061 ((S)-2-(5-nitrothiazol-2-ylcarbamoyl) phenyl 2-amino-3,3-dimethylbutanoate);
RM5064 ((S)-2-(5-chlorothiazol-2-ylcarbamoyl)phenyl 2-amino-3,3-dimethylbutanoate);
RM5066 ((2S,3S)-2-(5-nitrothiazol-2-ylcarbamoyl)phenyl 2-amino-3-methylpentanoate); and
RM5065 ((2S,3S)-2-(5-chlorothiazol-2-ylcarbamoyl)phenyl 2-amino-3-methyl pentanoate).

4. The method according to claim 1, wherein a pharmaceutical composition comprising said compound or a pharmaceutically acceptable salt thereof is administered to said subject.

5. The method according to claim 1, wherein the fibrotic disorder is selected from the group consisting of liver, gut, kidney, skin, epidermis, endodermis, muscle, tendon, cartilage, heart, pancreas, lung, uterus, nervous system, testis, penis, ovary, adrenal gland, artery, vein, colon, intestine (e.g., small intestine), biliary tract, soft tissue (e.g., mediastinum or retroperitoneum), bone marrow, joint, eye and stomach fibrosis.

6. The method according to claim 1, wherein the fibrotic disorder is selected from the group consisting of liver, gut, lung, heart, kidney, muscle, skin, soft tissue, bone marrow, intestinal, and joint fibrosis.

7. The method according to claim 1, wherein the fibrotic disorder is selected from the group consisting of non-alcoholic steatohepatitis (NASH), alcoholic steatohepatitis (ASH), pulmonary fibrosis, idiopathic pulmonary fibrosis, skin fibrosis, eye fibrosis, endomyocardial fibrosis, mediastinal fibrosis, myelofibrosis, retroperitoneal fibrosis, progressive massive fibrosis, proliferative fibrosis, neoplastic fibrosis, lung fibrosis consecutive to chronic inflammatory airway disease (COPD, asthma, emphysema, smoker's lung, tuberculosis), alcohol or drug-induced liver fibrosis, infection-induced liver fibrosis, radiation or chemotherapeutic-induced fibrosis, nephrogenic systemic fibrosis, Crohn's disease, ulcerative colitis, keloid, old myocardial infarction, scleroderma/systemic sclerosis, arthrofibrosis, some forms of adhesive capsulitis, chronic fibrosing cholangiopathies such as Primary Sclerosing Cholangitis (PSC), Primary Biliary Cholangitis (PBC), biliary atresia, and familial intrahepatic cholestasis type 3 (PFIC3), peri-implantational fibrosis and asbestosis.

8. The method according to claim 1, wherein the cholestatic disorder is selected in the group consisting of primary biliary cholangitis (PBC), primary sclerosing cholangitis (PSC), Intrahepatic Cholestasis of Pregnancy, Progressive Familial Intrahepatic Cholestasis, Biliary atresia, Cholelithiasis, Infectious Cholangitis, Cholangitis associated with Langerhans cell histiocytosis, Alagille syndrome, Nonsyndromic ductal paucity, Drug-induced cholestasis, and Total parenteral nutrition-associated cholestasis.

9. The method according to claim 1, said method comprising administering a compound of formula (I), or a pharmaceutically acceptable salt thereof, in combination with at least one therapeutically active agent.

10. The method according to claim 9, wherein the at least one therapeutically active agent is selected from pirfenidone or receptor tyrosine kinase inhibitors (RTKIs) such as Nintedanib, Sorafenib and other RTKIs, or angiotensin II (AT1) receptor blockers, or CTGF inhibitor, or any antifibrotic compound susceptible to interfere with the TGFβ and BMP-activated pathways including activators of the latent TGFβ complex such as MMP2, MMP9, THBS1 or cell-surface integrins, TGFβ receptors type I (TGFBRI) or type II (TGFBRII) and their ligands such as TGFβ, Activin, inhibin, Nodal, anti-Müllerian hormone, GDFs or BMPs, auxiliary co-receptors (also known as type III receptors), or components of the SMAD-dependent canonical pathway including regulatory or inhibitory SMAD proteins, or members of the SMAD-independent or non-canonical pathways including various branches of MAPK signaling, TAK1, Rho-like GTPase signaling pathways, phosphatidylinositol-3 kinase/AKT pathways, TGFβ-induced EMT process, or canonical and non-canonical Hedgehog signaling pathways including Hh ligands or target genes, or any members of the WNT, or Notch pathways which are susceptible to influence TGFβ signaling.

11. The method according to claim 9, wherein the at least one therapeutically active agent is selected from statins, JAK/STAT inhibitors, other anti-inflammatory and/or immunosuppressant agents.

12. The method according to claim 11, wherein the at least one therapeutically active agent is selected from fluvastatin, atorvastatin, lovastatin, simvastatin, mevastatin, cerivastatin, pitavastatin, glucocorticoids, NSAIDS, cyclophosphamide, nitrosoureas, folic acid analogs, purine analogs, pyrimidine analogs, methotrexate, azathioprine, mercaptopurine, ciclosporin, myriocin, tacrolimus, sirolimus, mycophenolic acid derivatives, fingolimod and other sphingosine-1-phosphate receptor modulators, monoclonal and/or polyclonal antibodies against such targets as proinflammatory cytokines and proinflammatory cytokine receptors, T-cell receptor, and integrins.

13. The method according to claim 4, wherein said composition is formulated in the form of injectable suspensions, gels, oils, ointments, pills, suppositories, powders, gel caps, capsules, and aerosols.
